(19) United States
(12) Patent Application Publication
(10) Pub. No.: US 2022/0098150 A1
(43) Pub. Date: Mar. 31, 2022

(54) Novel Crystalline Forms

(71) Applicant: Biogen MA Inc., Cambridge, MA (US)

(72) Inventors: Nishanth Gopinathan, Cambridge, MA (US); Erwin Irdam, Cambridge, MA (US); William Kiesman, Cambridge, MA (US); Daw-long A. Kwok, Cambridge, MA (US); Yiqing Lin, Cambridge, MA (US); Frederick Osei-Yeboah, Cambridge, MA (US); Matthew Peterson, Cambridge, MA (US); Kenny Tran, Cambridge, MA (US); Kalyan Vasudevan, Cambridge, MA (US)

(21) Appl. No.: 17/322,382

(22) Filed: May 17, 2021

Related U.S. Application Data
(62) Division of application No. 16/610,817, filed on Nov. 4, 2019, now abandoned.
(60) Provisional application No. 62/508,830, filed on May 19, 2017.

(51) Int. Cl. C07D 207/16

(52) U.S. Cl. CPC C07D 207/16; C07B 2200/13

(57) ABSTRACT

The present invention is directed to novel crystalline forms of 5-(4-{[2-fluorophenyl)methyl]oxy}phenyl-prolinamide hydrochloride, to the use of said crystalline forms in treating diseases and conditions mediated by modulation of voltage-gated sodium channels, to compositions containing said crystalline forms and processes for their preparation.

### REFERENCE TO RELATED APPLICATIONS

This application claims the benefit of U.S. Provisional Application No. 62/508,830, filed May 19, 2017, the contents of which are fully incorporated by reference herein.

### FIELD OF THE INVENTION

The present invention is directed to novel crystalline forms of 5-(4-{[(2-fluorophenyl)methyl]oxy}phenyl)-prolinamide hydrochloride, to the use of said crystalline forms in treating diseases and conditions mediated by modulation of voltage-gated sodium channels, to compositions containing said crystalline forms and processes for their preparation.

### BACKGROUND

The hydrochloride salt of (2S, 5R)-5-(4-((2-fluorobenzyl)oxy)phenyl)pyrrolidine-2-carboxamide, herein referred to as the compound of formula (I):

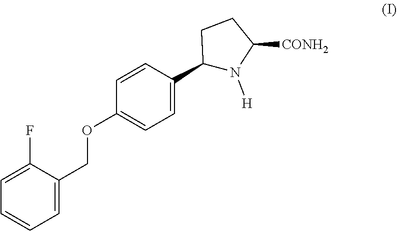 [SMILES: NC(=O)[C@@H]1CC[C@H](c2ccc(OCc3ccccc3F)cc2)N1]

is described in WO 2007/042239 as having utility in the treatment of diseases and conditions mediated by modulation of use-dependent voltage-gated sodium channels. The synthetic preparation of (2S, 5R)-5-(4-((2-fluorobenzyl)oxy)phenyl)pyrrolidine-2-carboxamide hydrochloride is described in both WO 2007/042239 and WO 2011/029762.

However, there is a need for the development of crystalline forms of such α-carboxamide pyrrolidine derivatives, which have desirable pharmaceutical properties.

### SUMMARY OF THE INVENTION

According to a first embodiment of the invention, there is provided a crystalline form of (5R)-5-(4-{[(2-fluorophenyl)methyl]oxy}phenyl)-L-prolinamide hydrochloride, characterised in that said crystalline form is either an anhydrous form or a solvated form.

According to a further embodiment of the invention, there is provided a pharmaceutical composition comprising the crystalline form as defined herein with one or more pharmaceutically acceptable carrier(s), diluents(s) and/or excipient(s).

According to a further embodiment of the invention, there is provided the crystalline form as defined herein for use in therapy.

According to a further embodiment of the invention, there is provided the crystalline form as defined herein for use in the treatment of a disease or condition mediated by modulation of voltage-gated sodium channels.

According to a further embodiment of the invention, there is provided the use of the crystalline form as defined herein in the manufacture of a medicament for the treatment of a disease or condition mediated by modulation of voltage-gated sodium channels.

According to a further embodiment of the invention, there is provided a method of treating a disease or condition mediated by modulation of voltage-gated sodium channels which comprises administering a therapeutically effective amount of the crystalline form as defined herein to a subject in need thereof.

### BRIEF DESCRIPTION OF THE FIGURES

FIG. 1: ORTEP representation of the compound of formula (I).H⁺Cl⁻ Form 1 (Anhydrous A) with thermal ellipsoids shown at 50% probability.

FIG. 2: pXRD pattern for the compound of formula (I).H⁺Cl⁻ Form 1 (Anhydrous A).

FIG. 3: ORTEP representation of the compound of formula (I).H⁺Cl⁻ Form 2 (Ethanol) with thermal ellipsoids shown at 50% probability.

FIG. 4: pXRD pattern for the compound of formula (I).H⁺Cl⁻ Form 2 (Ethanol).

FIG. 5: ORTEP representation of the compound of formula (I).H⁺Cl⁻ Form 3 (Methanol) with thermal ellipsoids shown at 50% probability. Note that one molecule of methanol is shown with 0.5 partial occupancy and that there are 1.5 molecules of methanol per compound of formula (I).

FIG. 6: pXRD pattern for the compound of formula (I).H⁺Cl⁻ Form 3 (Methanol).

FIG. 7: ORTEP representation of the compound of formula (I).H⁺Cl⁻ Form 4 (1-Propanol) with thermal ellipsoids shown at 50% probability.

FIG. 8: pXRD pattern for the compound of formula (I).H⁺Cl⁻ Form 4 (1-Propanol).

FIG. 9: ORTEP representation of the compound of formula (I).H⁺Cl⁻ Form 5 (1-Butanol) with thermal ellipsoids shown at 50% probability.

FIG. 10: pXRD pattern for the compound of formula (I).H⁺Cl⁻ Form 5 (1-Butanol).

FIG. 11: ORTEP representation of the compound of formula (I).H⁺Cl⁻ Form 6 (2-Methoxyethanol) with thermal ellipsoids shown at 50% probability.

FIG. 12: pXRD pattern for the compound of formula (I).H⁺Cl⁻ Form 6 (2-Methoxyethanol).

FIG. 13: ORTEP representation of the compound of formula (I).H⁺Cl⁻ Form 7 (Ethylene Glycol) with thermal ellipsoids shown at 50% probability. Note that the ethylene glycol molecule is disordered with partial site occupancy shown for clarity. It should also be noted that this figure shows a single representation of what is believed to be a number of disordered solvents.

FIG. 14: pXRD pattern for the compound of formula (I).H⁺Cl⁻ Form 7 (Ethylene Glycol).

FIG. 15: ORTEP representation of the compound of formula (I).H⁺Cl⁻ Form 8 (Propylene Glycol) with thermal ellipsoids shown at 50% probability. Note that the propylene glycol molecule and F1-containing ring are disordered with partial site occupancy shown for clarity.

FIG. 16: pXRD pattern for the compound of formula (I).H⁺Cl⁻ Form 8 (Propylene Glycol).

FIG. 17: ORTEP representation of the compound of formula (I).H⁺Cl⁻ Form 9 (Anhydrous B) with thermal ellipsoids shown at 50% probability.

FIG. 18: pXRD pattern for the compound of formula (I).H⁺Cl⁻ Form 9 (Anhydrous B).

FIG. 19: ORTEP representation of the compound of formula (I).H⁺Cl⁻ Form 10 (Anhydrous C) with thermal ellipsoids shown at 50% probability.

FIG. 20: pXRD pattern for the compound of formula (I).H⁺Cl⁻ Form 10 (Anhydrous C).

FIG. 21: Powder Bulk Density analysis of solid forms prepared according to the procedures described for Anhydrous Routes D, E and F.

FIG. 22: Powder Flow Function analysis of solid forms prepared according to the procedures described for Anhydrous Routes D, E and F.

FIG. 23: Powder Time Consolidation Behavior analysis of solid forms prepared according to the procedures described for Anhydrous Routes D, E and F.

### DETAILED DESCRIPTION OF THE INVENTION

According to a first embodiment of the invention, there is provided a crystalline form of (5R)-5-(4-{[(2-fluorophenyl)methyl]oxy}phenyl)-L-prolinamide hydrochloride, characterised in that said crystalline form is either an anhydrous form or a solvated form.

In one embodiment, the crystalline form is an anhydrous form. References herein to “anhydrous form” refer to solid forms that do not contain lattice water of crystallization. In a further embodiment, the anhydrous form is selected from anhydrous form A (Form 1), anhydrous form B (Form 9), or anhydrous form C (Form 10).

In one embodiment, the crystalline form is Anhydrous Form A (Form 1). Anhydrous Form A (Form 1) is the most stable crystalline form identified to date and advantageously demonstrates properties suitable for clinical development and commercial use.

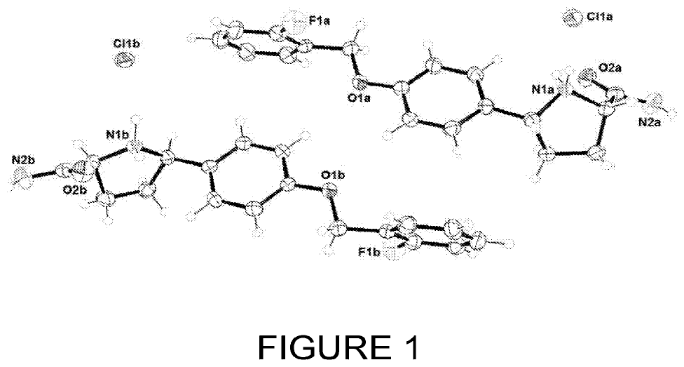

Anhydrous Form A (Form 1) is described herein in Example 2 and is depicted in FIG. 1. According to a further embodiment of the invention, there is provided a process for preparing Anhydrous Form A (Form 1) which comprises the methodology described in Example 2. In one embodiment, anhydrous form A (Form 1) is characterised by any one or more or all of the parameters in Table 1.

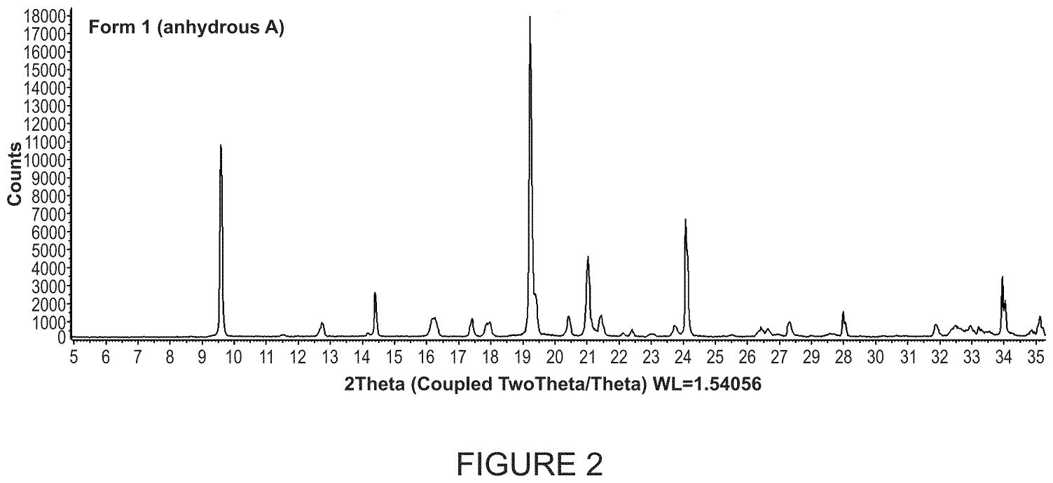

In a further embodiment, the anhydrous form A (Form 1) is characterised by an X-ray diffraction pattern having 28 Diffraction (°) peaks at: 9.56, 11.48, 12.71, 14.30, 16.23, 17.49, 17.87, 19.23, 19.74, 19.87, 20.40, 21.09, 21.47, 22.47, 23.06, 23.87, 24.10, 26.61, 26.79, 27.37, 28.09, 31.89, 32.66, 33.25 and 34.20. These peaks relate to those extrapolated from the X-ray diffraction pattern of FIG. 2.

In a further embodiment, the anhydrous form A (Form 1) is characterised by an X-ray diffraction pattern having 2θ Diffraction (°) peaks at: 9.56, 12.71, 19.23, 20.40, 21.09, 21.47 and 27.37. These peaks relate to the strongest peaks extrapolated from the X-ray diffraction pattern of FIG. 2.

In a further embodiment, the anhydrous form A (Form 1) is characterised by the X-ray diffraction pattern of FIG. 2.

In one embodiment, the crystalline form is Anhydrous Form B (Form 9). Anhydrous Form B (Form 9) is less stable than Anhydrous Form A (Form 1) but may have the advantage of possessing higher solubility than Anhydrous Form A (Form 1).

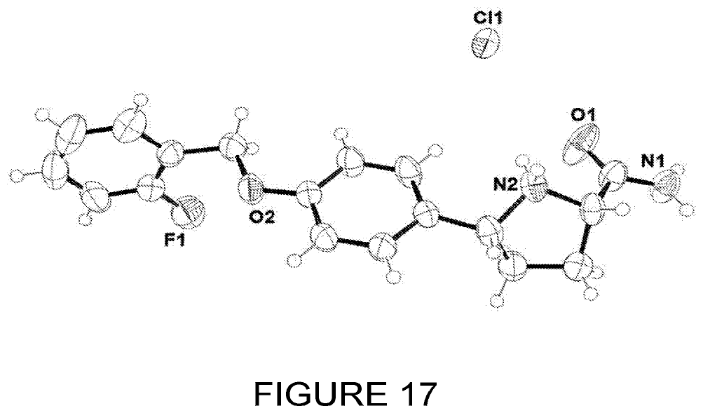

Anhydrous Form B (Form 9) is described herein in Example 10 and is depicted in FIG. 17. According to a further embodiment of the invention, there is provided a process for preparing Anhydrous Form B (Form 9) which comprises the methodology described in Example 10. In one embodiment, anhydrous form B (Form 9) is characterised by any one or more or all of the parameters in Table 17.

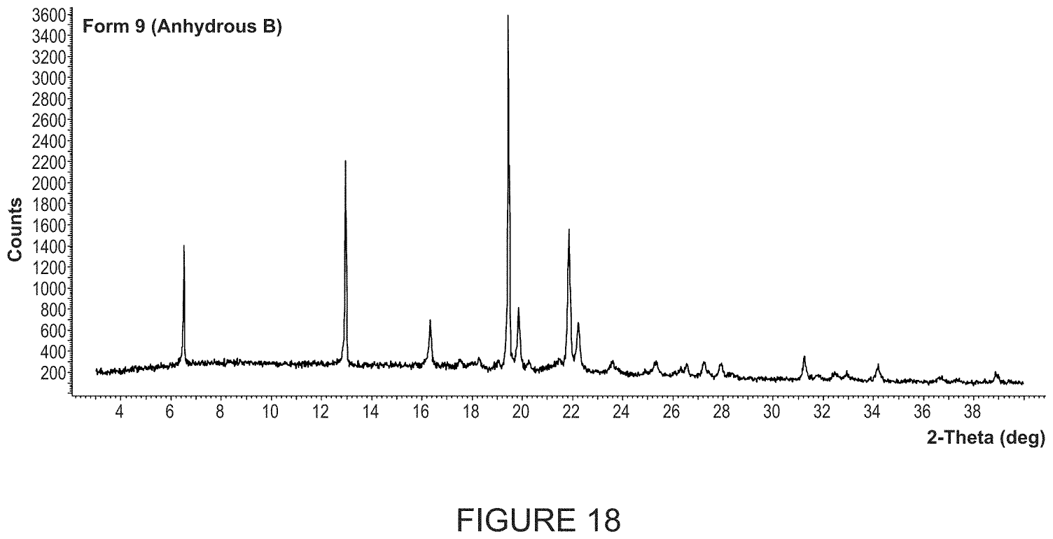

In a further embodiment, the anhydrous form B (Form 9) is characterised by an X-ray diffraction pattern having 2θ Diffraction (°) peaks at: 6.52, 12.95, 16.33, 19.44, 19.85, 21.86, 22.23, 23.56, 25.27, 26.51, 27.21 and 27.86. These peaks relate to those extrapolated from the X-ray diffraction pattern of FIG. 18.

In a further embodiment, the anhydrous form B (Form 9) is characterised by an X-ray diffraction pattern having 2e Diffraction (°) peaks at: 16.33 and 21.86. These peaks relate to the strongest peaks extrapolated from the X-ray diffraction pattern of FIG. 18.

In a further embodiment, the anhydrous form B (Form 9) is characterised by an X-ray diffraction pattern having a 2θ Diffraction (°) peak at 6.52. This peak relates to a differentiating peak between the X-ray diffraction pattern of FIG. 18 and the X-ray diffraction pattern of Anhydrous Form A (Form 1) in FIG. 2.

In a further embodiment, the anhydrous form B (Form 9) is characterised by the X-ray diffraction pattern of FIG. 18.

In one embodiment, the crystalline form is Anhydrous Form C (Form 10). Anhydrous Form C (Form 10) is less stable than Anhydrous Form A (Form 1) but may have the advantage of possessing higher solubility than Anhydrous Form A (Form 1).

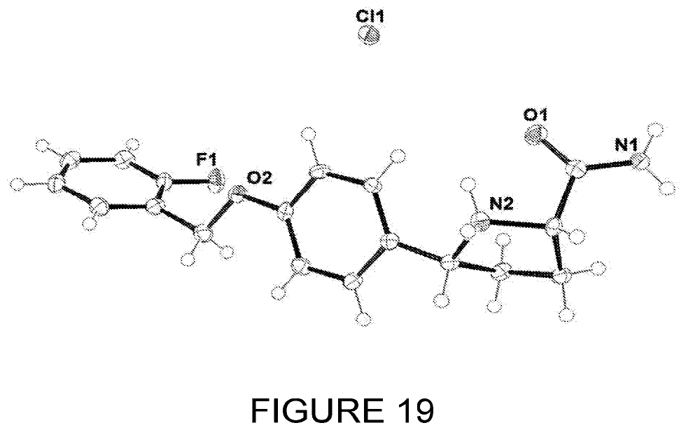

Anhydrous Form C (Form 10) is described herein in Example 11 and is depicted in FIG. 19. According to a further embodiment of the invention, there is provided a process for preparing Anhydrous Form C (Form 10) which comprises the methodology described in Example 11. In one embodiment, anhydrous form C (Form 10) is characterised by any one or more or all of the parameters in Table 19.

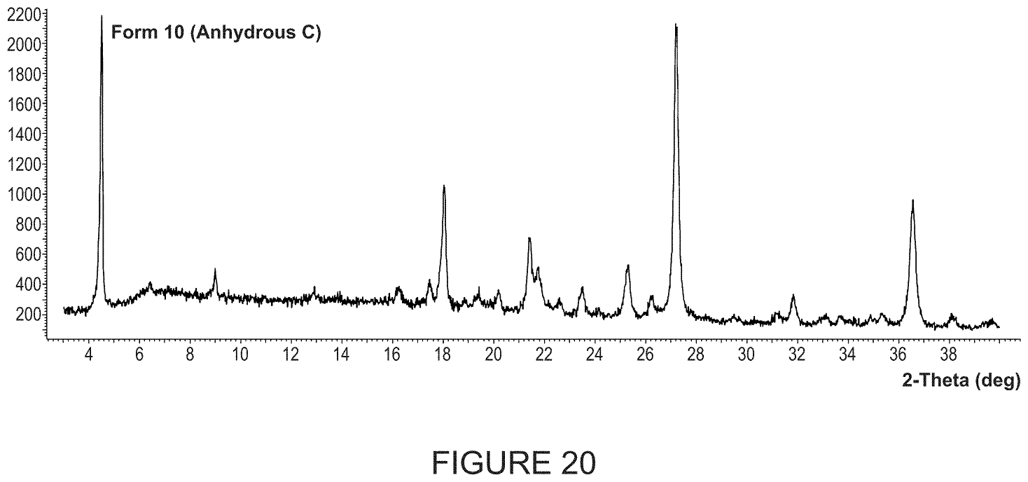

In one embodiment, the anhydrous form C (Form 10) is characterised by an X-ray diffraction pattern having 2θ Diffraction (°) peaks at: 4.51, 8.99, 12.97, 17.48, 18.03, 19.45, 20.19, 21.39, 21.76, 23.50, 25.34, 26.37, 27.19, 31.84, 33.14 and 36.57. These peaks relate to those extrapolated from the X-ray diffraction pattern of FIG. 20.

In a further embodiment, the anhydrous form C (Form 10) is characterised by an X-ray diffraction pattern having 2θ Diffraction (°) peaks at: 17.48, 20.19, 21.76, 23.50 and 26.37. These peaks relate to the strongest peaks extrapolated from the X-ray diffraction pattern of FIG. 20.

In a further embodiment, the anhydrous form C (Form 10) is characterised by an X-ray diffraction pattern having 2θ Diffraction (°) peaks at: 4.51, 8.99 and 18.03. These peaks relate to differentiating peaks between the X-ray diffraction pattern of FIG. 20 and the X-ray diffraction pattern of Anhydrous Form A (Form 1) in FIG. 2.

In a further embodiment, the anhydrous form C (Form 10) is characterised by the X-ray diffraction pattern of FIG. 20.

In one embodiment, the crystalline form is a solvated form. The term “solvated form” refers to solid forms in which solvent is incorporated into the crystal lattice. This physical association may involve varying degrees of ionic and covalent bonding, including hydrogen bonding. The term “solvate” is intended to encompass both solution-phase and isolated solvates. In a further embodiment, the crystalline form is a form solvated with ethanol, methanol, 1-propanol, 1-butanol, 2-methoxyethanol, ethylene glycol, or propylene glycol.

In a further embodiment, the crystalline form is the ethanol solvate (Form 2). The ethanol solvate (Form 2) is believed to find utility as a potential processing intermediate and therefore represents an alternative synthetic route to isolating Anhydrous Form A (Form 1). Thus, according to a further embodiment of the invention there is provided the use of the ethanol solvate (Form 2) as an intermediate in the preparation of Anhydrous Form A (Form 1).

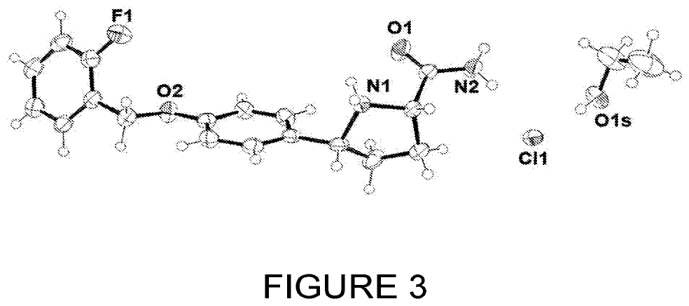

The ethanol solvate (Form 2) is described herein in Example 3 and is depicted in FIG. 3. According to a further embodiment of the invention, there is provided a process for preparing the ethanol solvate (Form 2) which comprises the methodology described in Example 3. In one embodiment, the ethanol solvate (Form 2) is characterised by any one or more or all of the parameters in Table 3.

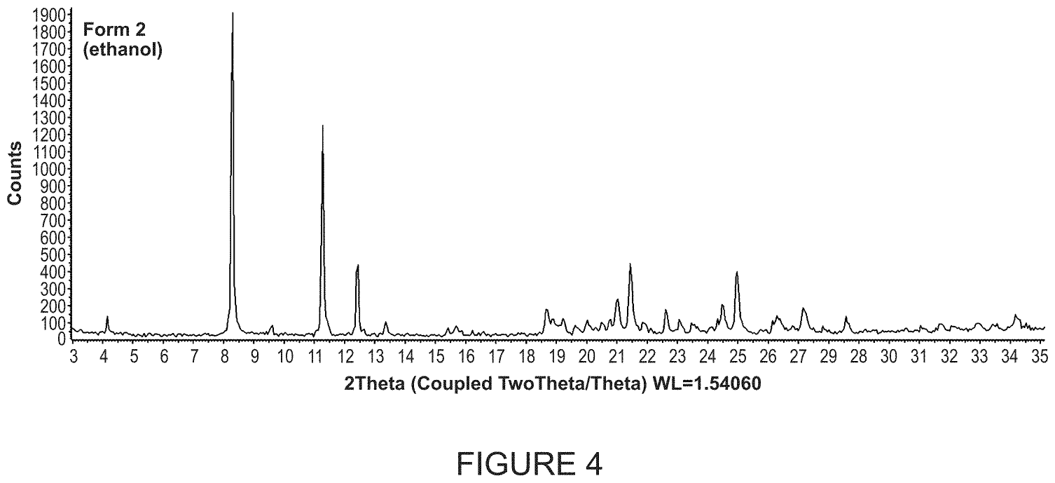

In one embodiment, the ethanol solvate (Form 2) is characterised by an X-ray diffraction pattern having 29 Diffraction (°) peaks at: 4.16, 8.31, 11.29, 12.45, 13.36, 15.43, 15.69, 16.24, 18.67, 18.92, 20.03, 20.49, 21.04, 21.45, 22.05, 22.61, 23.07, 23.57, 24.48, 26.30, 27.16 and 28.57. These peaks relate to those extrapolated from the X-ray diffraction pattern of FIG. 4.

In a further embodiment, the ethanol solvate (Form 2) is characterised by an X-ray diffraction pattern having 2θ Diffraction (°) peaks at: 8.31, 11.29, 18.67, 21.45 and 27.16. These peaks relate to the strongest peaks extrapolated from the X-ray diffraction pattern of FIG. 4.

In a further embodiment, the ethanol solvate (Form 2) is characterised by an X-ray diffraction pattern having 29 Diffraction (°) peaks at: 4.16, 8.31, 13.36 and 15.43. These peaks relate to differentiating peaks between the X-ray diffraction pattern of FIG. 4 and the X-ray diffraction pattern of Anhydrous Form A (Form 1) in FIG. 2.

In a further embodiment, the ethanol solvate (Form 2) is characterised by an X-ray diffraction pattern having a 2θ Diffraction (°) peak at: 8.31. This peak relates to the strongest peak extrapolated from the X-ray diffraction pattern of FIG. 4 which also provides a differentiating peak between the X-ray diffraction pattern of FIG. 4 and the X-ray diffraction pattern of Anhydrous Form A (Form 1) in FIG. 2.

In a further embodiment, the ethanol solvate (Form 2) is characterised by the X-ray diffraction pattern of FIG. 4.

In a further embodiment, the crystalline form is the methanol solvate (Form 3). The methanol solvate (Form 3) is believed to find utility as a potential processing intermediate and therefore represents an alternative synthetic route to isolating Anhydrous Form A (Form 1). Thus, according to a further embodiment of the invention there is provided the use of the methanol solvate (Form 3) as an intermediate in the preparation of Anhydrous Form A (Form 1).

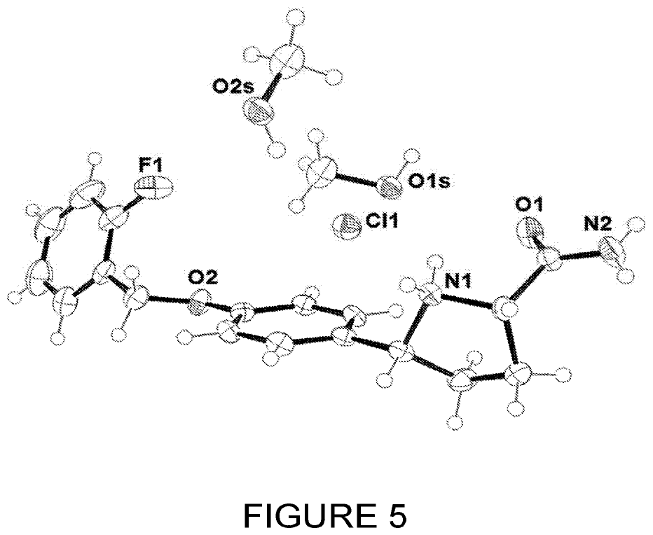

The methanol solvate (Form 3) is described herein in Example 4 and is depicted in FIG. 5. According to a further embodiment of the invention, there is provided a process for preparing the methanol solvate (Form 3) which comprises the methodology described in Example 4. In one embodiment, the methanol solvate (Form 3) is characterised by any one or more or all of the parameters in Table 5.

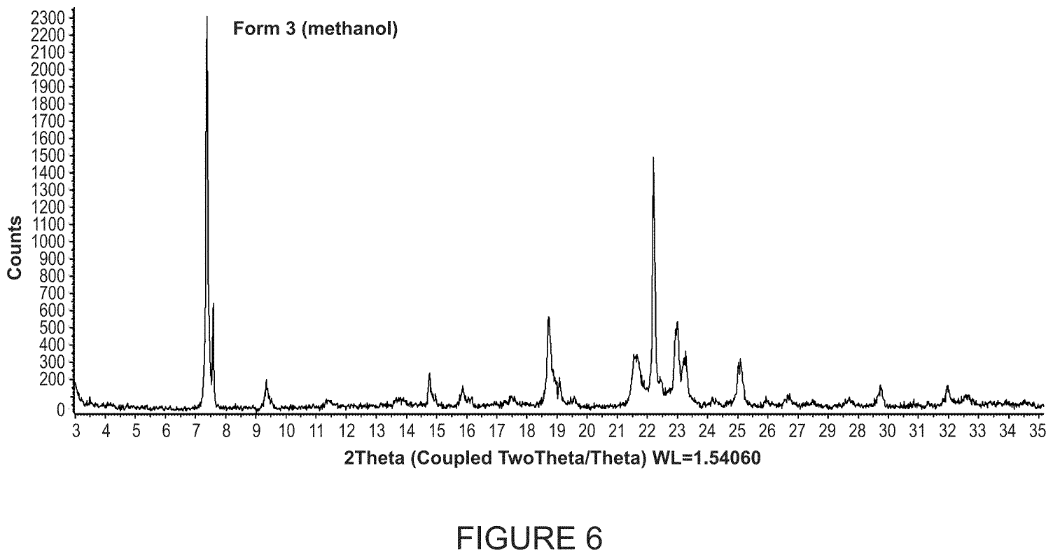

In one embodiment, the methanol solvate (Form 3) is characterised by an X-ray diffraction pattern having 2θ Diffraction (°) peaks at: 7.55, 9.53, 14.98, 16.05, 17.70, 18.85, 19.30, 21.94, 22.45, 22.79, 23.30, 24.18, 25.23, 26.07, 26.60, 27.61, 28.76, 29.62, 31.00, 32.20 and 32.91. These peaks relate to those extrapolated from the X-ray diffraction pattern of FIG. 6.

In a further embodiment, the methanol solvate (Form 3) is characterised by an X-ray diffraction pattern having 2e Diffraction (°) peaks at: 7.55, 18.85, 19.30, 22.45 and 23.30. These peaks relate to the strongest peaks extrapolated from the X-ray diffraction pattern of FIG. 6.

In a further embodiment, the methanol solvate (Form 3) is characterised by an X-ray diffraction pattern having 2θ Diffraction (°) peaks at: 7.55, 14.98 and 29.62. These peaks relate to differentiating peaks between the X-ray diffraction pattern of FIG. 6 and the X-ray diffraction pattern of Anhydrous Form A (Form 1) in FIG. 2.

In a further embodiment, the methanol solvate (Form 3) is characterised by an X-ray diffraction pattern having a 28 Diffraction (°) peak at 7.55. This peak relates to the strongest peak extrapolated from the X-ray diffraction pattern of FIG. 6 which also provides a differentiating peak between the X-ray diffraction pattern of FIG. 6 and the X-ray diffraction pattern of Anhydrous Form A (Form 1) in FIG. 2.

In a further embodiment, the methanol solvate (Form 3) is characterised by the X-ray diffraction pattern of FIG. 6.

In a further embodiment, the crystalline form is the 1-propanol solvate (Form 4). The 1-propanol solvate (Form 4) is believed to find utility as a potential processing intermediate and therefore represents an alternative synthetic route to isolating Anhydrous Form A (Form 1). Thus, according to a further embodiment of the invention there is provided the use of the 1-propanol solvate (Form 4) as an intermediate in the preparation of Anhydrous Form A (Form 1).

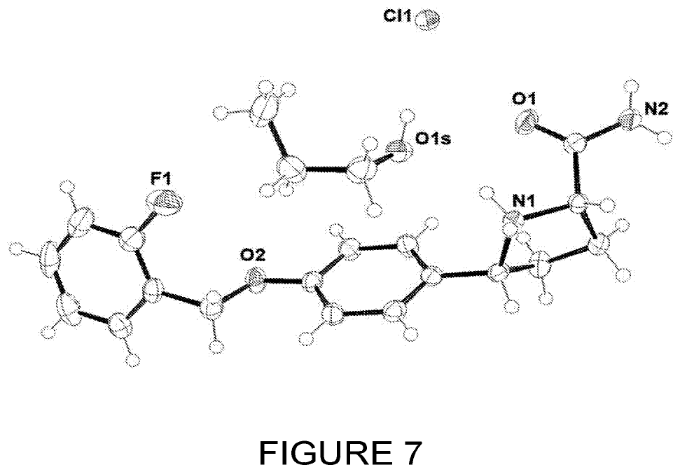

The 1-propanol solvate (Form 4) is described herein in Example 5 and is depicted in FIG. 7. According to a further embodiment of the invention, there is provided a process for preparing the 1-propanol solvate (Form 4) which comprises the methodology described in Example 5. In one embodiment, the 1-propanol solvate (Form 4) is characterised by any one or more or all of the parameters in Table 7.

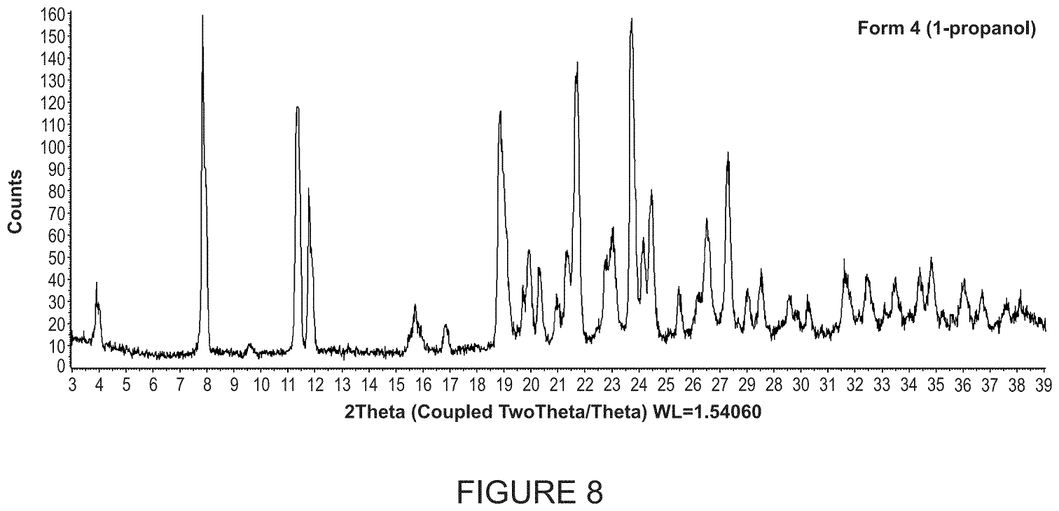

In one embodiment, the 1-propanol solvate (Form 4) is characterised by an X-ray diffraction pattern having 2θ Diffraction (°) peaks at: 3.92, 7.85, 11.37, 11.78, 15.82, 16.94, 18.92, 20.91, 21.72, 22.97, 23.77, 24.13, 24.47, 25.46, 26.17, 28.15, 31.66 and 34.84. These peaks relate to those extrapolated from the X-ray diffraction pattern of FIG. 8.

In a further embodiment, the 1-propanol solvate (Form 4) is characterised by an X-ray diffraction pattern having 2θ Diffraction (°) peaks at: 7.85, 11.37, 18.92, 21.72 and 22.97. These peaks relate to the strongest peaks extrapolated from the X-ray diffraction pattern of FIG. 8.

In a further embodiment, the 1-propanol solvate (Form 4) is characterised by an X-ray diffraction pattern having 2θ Diffraction (°) peaks at: 3.92 and 7.85. These peaks relate to differentiating peaks between the X-ray diffraction pattern of FIG. 8 and the X-ray diffraction pattern of Anhydrous Form A (Form 1) in FIG. 2.

In a further embodiment, the 1-propanol solvate (Form 4) is characterised by an X-ray diffraction pattern having a 2θ Diffraction (°) peak at 7.85. This peak relates to the strongest peak extrapolated from the X-ray diffraction pattern of FIG. 8 which also provides a differentiating peak between the X-ray diffraction pattern of FIG. 8 and the X-ray diffraction pattern of Anhydrous Form A (Form 1) in FIG. 2.

In a further embodiment, the 1-propanol solvate (Form 4) is characterised by the X-ray diffraction pattern of FIG. 8.

In a further embodiment, the crystalline form is the 1-butanol solvate (Form 5). The 1-butanol solvate (Form 5) is believed to find utility as a potential processing intermediate and therefore represents an alternative synthetic route to isolating Anhydrous Form A (Form 1). Thus, according to a further embodiment of the invention there is provided the use of the 1-butanol solvate (Form 5) as an intermediate in the preparation of Anhydrous Form A (Form 1).

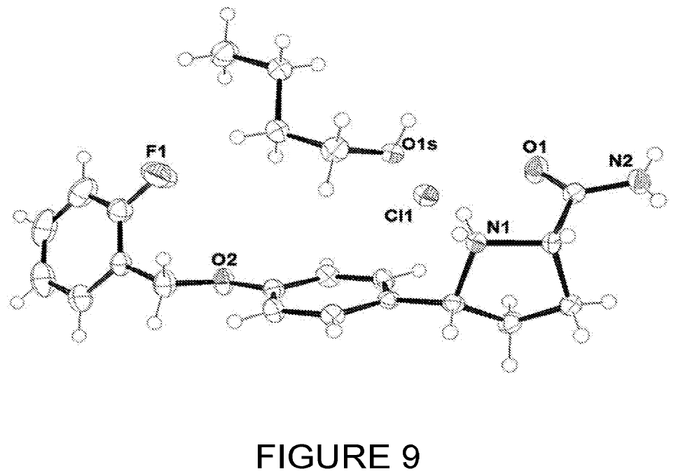

The 1-butanol solvate (Form 5) is described herein in Example 6 and is depicted in FIG. 9. According to a further embodiment of the invention, there is provided a process for preparing the 1-butanol solvate (Form 5) which comprises the methodology described in Example 6. In one embodiment, the 1-butanol solvate (Form 5) is characterised by any one or more or all of the parameters in Table 9.

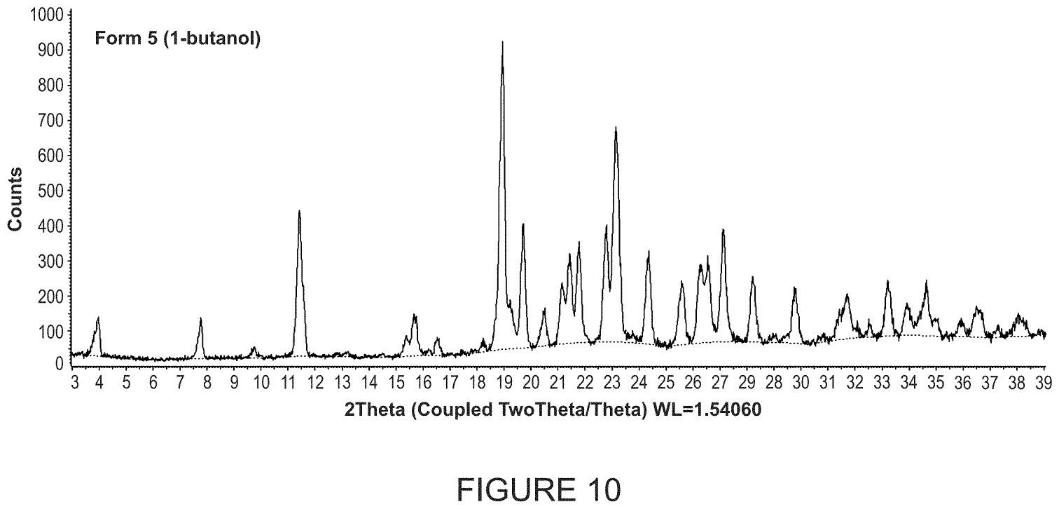

In one embodiment, the 1-butanol solvate (Form 5) is characterised by an X-ray diffraction pattern having 2θ Diffraction (°) peaks at: 3.92, 7.78, 11.45, 15.57, 15.72, 16.56, 18.95, 19.74, 21.24, 21.53, 21.88, 23.14, 24.43, 25.54, 26.35, 27.20, 28.32, 31.74, 33.37 and 34.66. These peaks relate to those extrapolated from the X-ray diffraction pattern of FIG. 10.

In a further embodiment, the 1-butanol solvate (Form 5) is characterised by an X-ray diffraction pattern having 2θ Diffraction (°) peaks at: 11.45, 18.95 and 23.14. These peaks relate to the strongest peaks extrapolated from the X-ray diffraction pattern of FIG. 10.

In a further embodiment, the 1-butanol solvate (Form 5) is characterised by an X-ray diffraction pattern having 2θ Diffraction (°) peaks at: 3.92 and 7.78. These peaks relate to differentiating peaks between the X-ray diffraction pattern of FIG. 10 and the X-ray diffraction pattern of Anhydrous Form A (Form 1) in FIG. 2.

In a further embodiment, the 1-butanol solvate (Form 5) is characterised by the X-ray diffraction pattern of FIG. 10.

In a further embodiment, the crystalline form is the 2-methoxyethanol solvate (Form 6). The 2-methoxyethanol solvate (Form 6) is believed to find utility as a potential processing intermediate and therefore represents an alternative synthetic route to isolating Anhydrous Form A (Form 1). Thus, according to a further embodiment of the invention there is provided the use of the 2-methoxyethanol solvate (Form 6) as an intermediate in the preparation of Anhydrous Form A (Form 1).

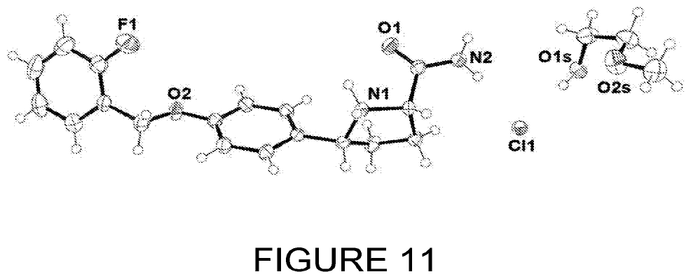

The 2-methoxyethanol solvate (Form 6) is described herein in Example 7 and is depicted in FIG. 11. According to a further embodiment of the invention, there is provided a process for preparing the 2-methoxyethanol solvate (Form 6) which comprises the methodology described in Example 7. In one embodiment, the 2-methoxyethanol solvate (Form 6) is characterised by any one or more or all of the parameters in Table 11.

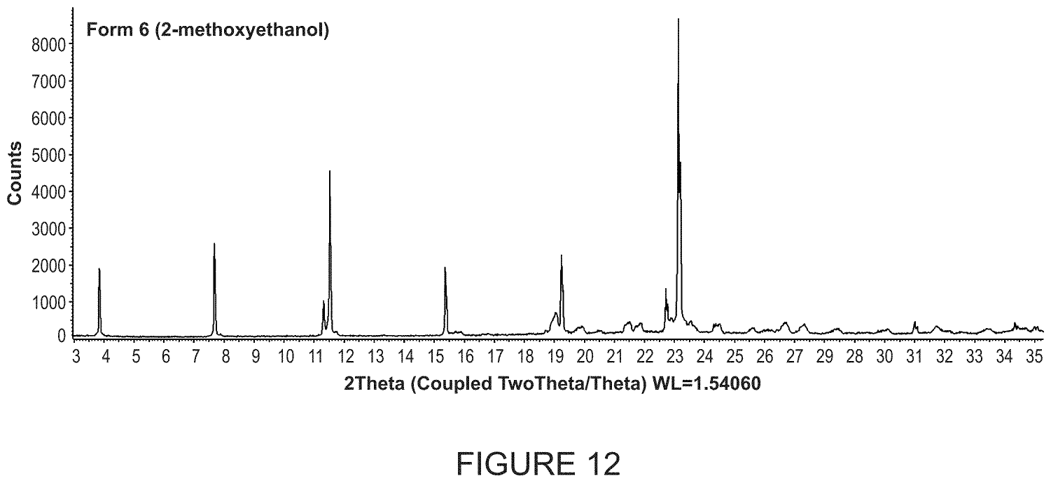

In one embodiment, the 2-methoxyethanol solvate (Form 6) is characterised by an X-ray diffraction pattern having 2θ Diffraction (°) peaks at: 3.86, 7.70, 11.54, 15.38, 19.05, 19.30, 19.96, 21.56, 21.90, 23.17, 24.51, 25.53 and 31.79. These peaks relate to those extrapolated from the X-ray diffraction pattern of FIG. 12.

In a further embodiment, the 2-methoxyethanol solvate (Form 6) is characterised by an X-ray diffraction pattern having 2θ Diffraction (°) peaks at: 11.54, 19.05 and 23.17. These peaks relate to the strongest peaks extrapolated from the X-ray diffraction pattern of FIG. 12.

In a further embodiment, the 2-methoxyethanol solvate (Form 6) is characterised by an X-ray diffraction pattern having 28 Diffraction (°) peaks at: 3.86 and 7.70. These peaks relate to differentiating peaks between the X-ray diffraction pattern of FIG. 12 and the X-ray diffraction pattern of Anhydrous Form A (Form 1) in FIG. 2.

In a further embodiment, the 2-methoxyethanol solvate (Form 6) is characterised by the X-ray diffraction pattern of FIG. 12.

In a further embodiment, the crystalline form is the ethylene glycol solvate (Form 7). The ethylene glycol solvate (Form 7) is believed to find utility as a potential processing intermediate and therefore represents an alternative synthetic route to isolating Anhydrous Form A (Form 1). Thus, according to a further embodiment of the invention there is provided the use of the ethylene glycol solvate (Form 7) as an intermediate in the preparation of Anhydrous Form A (Form 1).

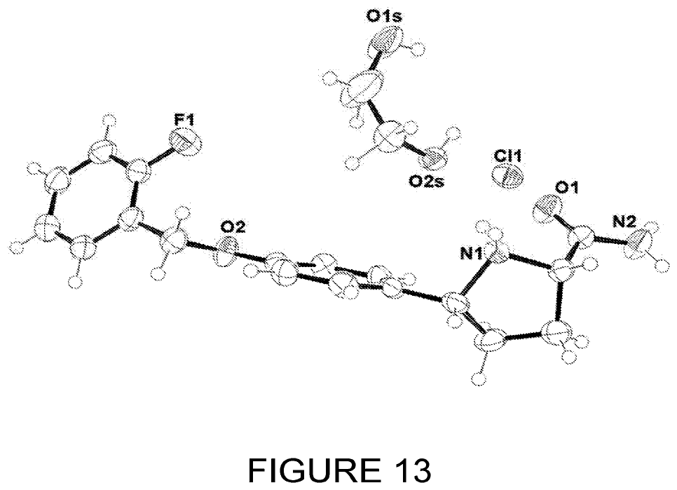

The ethylene glycol solvate (Form 7) is described herein in Example 8 and is depicted in FIG. 13. According to a further embodiment of the invention, there is provided a process for preparing the ethylene glycol solvate (Form 7) which comprises the methodology described in Example 8. In one embodiment, the ethylene glycol solvate (Form 7) is characterised by any one or more or all of the parameters in Table 13.

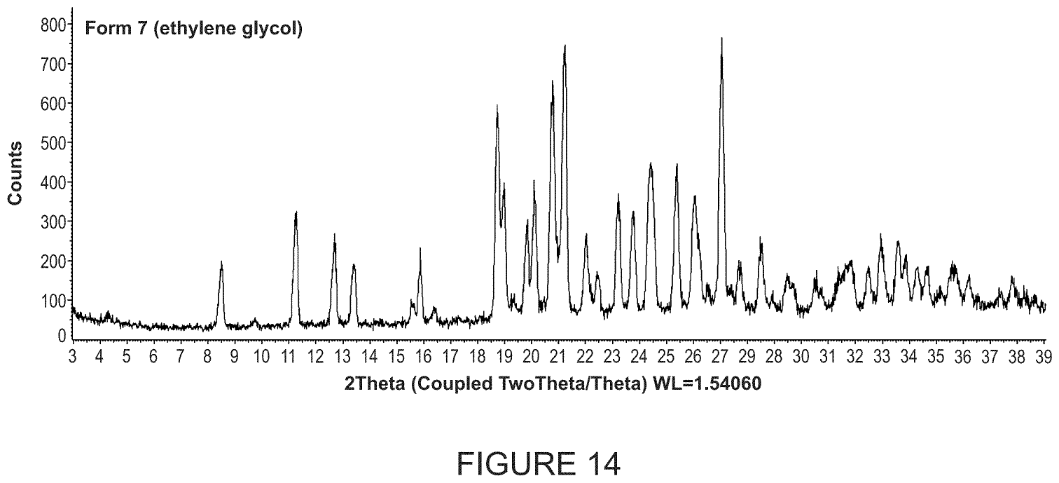

In one embodiment, the ethylene glycol solvate (Form 7) is characterised by an X-ray diffraction pattern having 2θ Diffraction (°) peaks at: 8.38, 11.29, 12.69, 13.40, 15.54, 15.89, 16.40, 18.74, 18.95, 19.79, 20.12, 20.73, 21.24, 21.90, 22.43, 23.26, 23.78, 24.43, 26.35, 26.02, 27.06, 27.71, 28.50, 29.47, 29.68, 30.51, 30.66, 32.96, 33.57, 33.89, 35.75 and 37.86. These peaks relate to those extrapolated from the X-ray diffraction pattern of FIG. 14.

In a further embodiment, the ethylene glycol solvate (Form 7) is characterised by an X-ray diffraction pattern having 29 Diffraction (°) peaks at: 11.29, 18.74, 18.95, 20.73, 21.24, 24.43, 26.35 and 27.06. These peaks relate to the strongest peaks extrapolated from the X-ray diffraction pattern of FIG. 14.

In a further embodiment, the ethylene glycol solvate (Form 7) is characterised by an X-ray diffraction pattern having 29 Diffraction (°) peaks at: 8.38, 13.40, 18.74, 18.95 and 29.68. These peaks relate to differentiating peaks between the X-ray diffraction pattern of FIG. 14 and the X-ray diffraction pattern of Anhydrous Form A (Form 1) in FIG. 2.

In a further embodiment, the ethylene glycol solvate (Form 7) is characterised by an X-ray diffraction pattern having a 2θ Diffraction (°) peak at 18.74. This peak relates to the strongest peak extrapolated from the X-ray diffraction pattern of FIG. 14 which also provides a differentiating peak between the X-ray diffraction pattern of FIG. 14 and the X-ray diffraction pattern of Anhydrous Form A (Form 1) in FIG. 2.

In a further embodiment, the ethylene glycol solvate (Form 7) is characterised by the X-ray diffraction pattern of FIG. 14.

In a further embodiment, the crystalline form is the propylene glycol solvate (Form 8). The propylene glycol solvate (Form 8) is believed to find utility as a potential processing intermediate and therefore represents an alternative synthetic route to isolating Anhydrous Form A (Form 1). Thus, according to a further embodiment of the invention there is provided the use of the propylene glycol solvate (Form 8) as an intermediate in the preparation of Anhydrous Form A (Form 1).

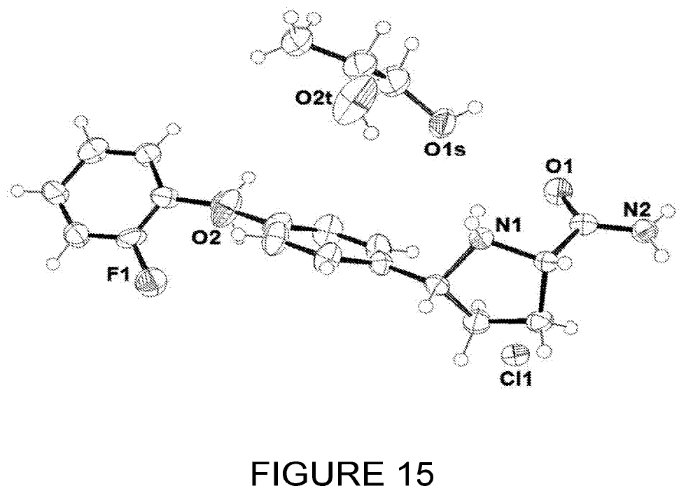

The propylene glycol solvate (Form 8) is described herein in Example 9 and is depicted in FIG. 15. According to a further embodiment of the invention, there is provided a process for preparing the propylene glycol solvate (Form 8) which comprises the methodology described in Example 9. In one embodiment, the propylene glycol solvate (Form 8) is characterised by any one or more or all of the parameters in Table 15.

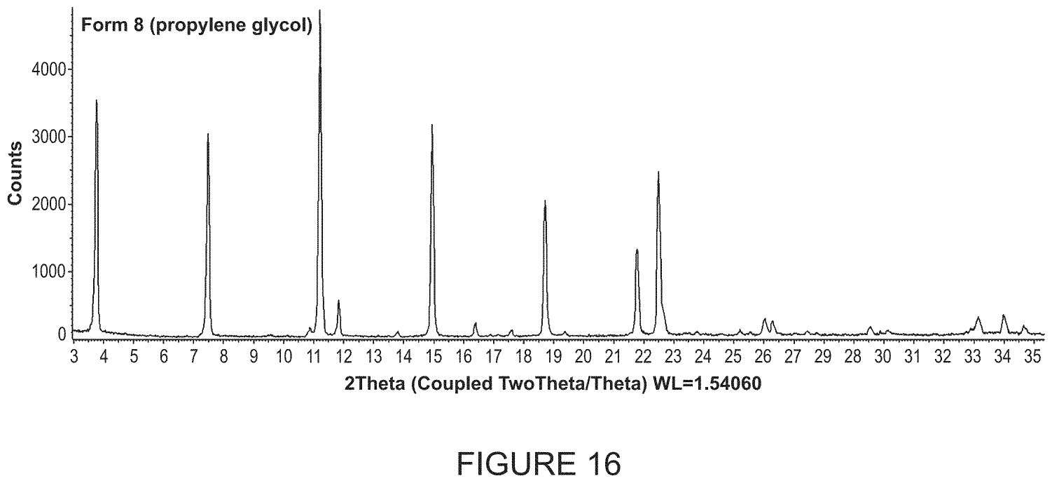

In one embodiment, the propylene glycol solvate (Form 8) is characterised by an X-ray diffraction pattern having 28 Diffraction (°) peaks at: 7.47, 10.86, 11.21, 11.85, 13.80, 14.95, 16.42, 16.86, 17.59, 18.71, 21.80, 22.48, 25.22, 25.46 and 27.06. These peaks relate to those extrapolated from the X-ray diffraction pattern of FIG. 16.

In a further embodiment, the propylene glycol solvate (Form 8) is characterised by an X-ray diffraction pattern having 28 Diffraction (°) peaks at: 11.85, 16.86 and 21.80. These peaks relate to the strongest peaks extrapolated from the X-ray diffraction pattern of FIG. 16.

In a further embodiment, the propylene glycol solvate (Form 8) is characterised by an X-ray diffraction pattern having 2θ Diffraction (°) peaks at: 7.47, 11.85 and 14.95. These peaks relate to differentiating peaks between the X-ray diffraction pattern of FIG. 16 and the X-ray diffraction pattern of Anhydrous Form A (Form 1) in FIG. 2.

In a further embodiment, the propylene glycol solvate (Form 8) is characterised by an X-ray diffraction pattern having a 28 Diffraction (°) peak at 11.85. This peak relates to the strongest peak extrapolated from the X-ray diffraction pattern of FIG. 16 which also provides a differentiating peak between the X-ray diffraction pattern of FIG. 16 and the X-ray diffraction pattern of Anhydrous Form A (Form 1) in FIG. 2.

In a further embodiment, the propylene glycol solvate (Form 8) is characterised by the X-ray diffraction pattern of FIG. 16.

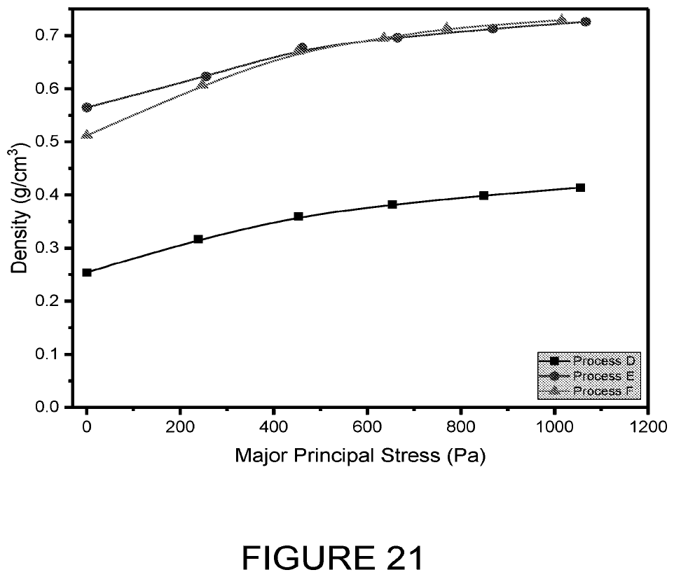

In one embodiment, the crystalline form defined herein is an anhydrous form selected from any one of the solid forms of Examples 12-14. Data is presented herein in Table 21 and FIGS. 21-23 which demonstrate beneficial properties of the solid forms of Routes E and F and a solid form of anhydrous Route D as active pharmaceutical ingredients.

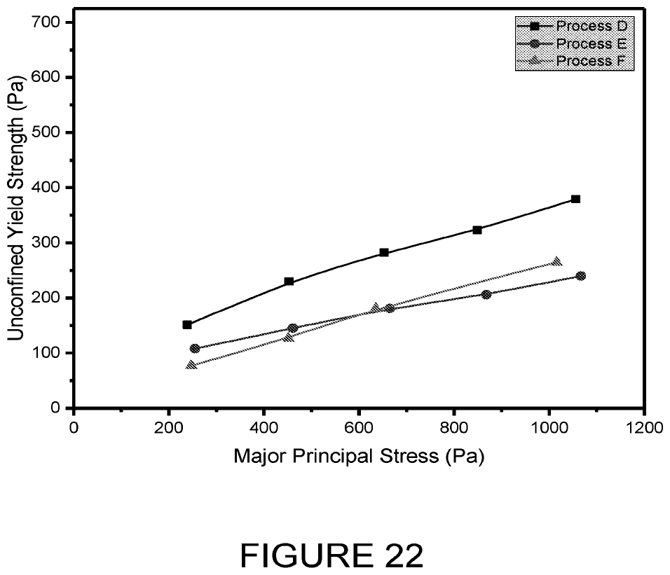

In a further embodiment, the crystalline form defined herein is an anhydrous form selected from any one of the solid forms of Examples 13-14. Data is presented herein in Table 21 and FIGS. 21-23 which demonstrate superior beneficial properties of the products of these routes (i.e. Routes E and F) as active pharmaceutical ingredients compared with a product of anhydrous Route D described in Example 12, in particular with respect to powder bulk density (see FIG. 21) and powder flow functions (see FIG. 22).

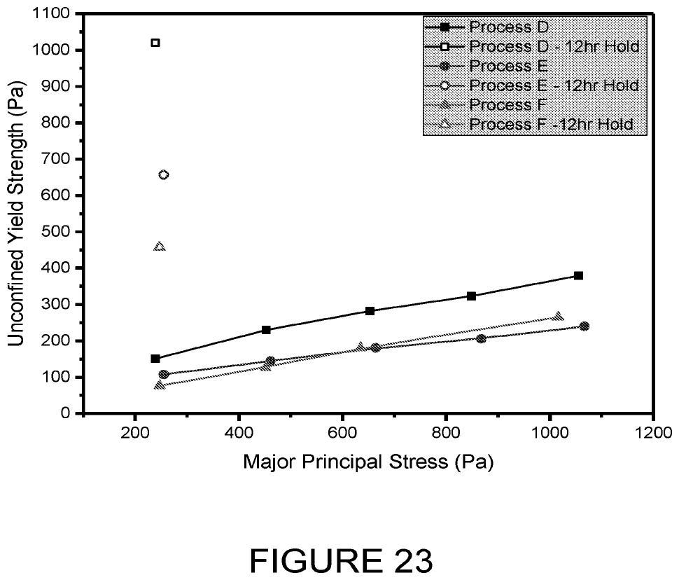

In a yet further embodiment, the crystalline form defined herein is an anhydrous form of Example 13. Data is presented herein in Table 21 and FIGS. 21-23 which demonstrate superior beneficial properties of the product of this route (i.e. Route E) as an active pharmaceutical ingredient compared with the product of Route F (Example 14) and the product of anhydrous Route D, in particular with respect to powder bulk density (see FIG. 21), powder flow functions (see FIG. 22), and powder time consolidation behavior (see FIG. 23).

According to a further embodiment of the invention, there is provided an anhydrous crystalline form of (5R)-5-(4-{[(2-fluorophenyl)methyl]oxy}phenyl)-L-prolinamide hydrochloride, characterised in that said anhydrous crystalline form has an initial bulk density, tested as defined herein, of at least 0.4 g/cm³, such as at least 0.5 g/cm³.

According to a further embodiment of the invention, there is provided an anhydrous crystalline form of (5R)-5-(4-{[(2-fluorophenyl)methyl]oxy}phenyl)-L-prolinamide hydrochloride, characterised in that said anhydrous crystalline form has an unconfined yield strength of less than 200 Pa at a major principal stress value of 500 Pa, tested in accordance with the powder flow function analysis herein.

As discussed hereinabove, it is believed that crystalline forms of the invention (herein also referred to as the compounds of the invention), in particular Anhydrous Form A (Form 1), Anhydrous Form B (Form 9) and Anhydrous Form C (Form 10) may be useful for the treatment of diseases and conditions mediated by modulation of voltage-gated sodium channels.

In one embodiment, the compounds will be state-dependent sodium channel inhibitors.

In another embodiment, the compounds will be subtype NaV1.7 sodium channel state-dependent inhibitors.

In another embodiment, the compounds will be state-dependent sodium channel inhibitors which have a suitable developability profile on oral administration, for example in terms of exposure (Cmax) and/or bioavailability.

In one embodiment, the compounds will be sodium channel inhibitors.

In another embodiment, the compounds will be subtype NaV1.7 sodium channel inhibitors.

In another embodiment, the compounds will be sodium channel inhibitors which have a suitable developability profile on oral administration, for example in terms of exposure (Cmax) and/or bioavailability.

According to a further embodiment of the invention, there is provided compounds of the invention for use as a medicament, preferably a human medicament.

According to a further embodiment the invention provides the use of compounds of the invention in the manufacture of a medicament for treating or preventing a disease or condition mediated by modulation of voltage-gated sodium channels.

In one particular embodiment, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis); musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the amelioration of inflammatory disorders, for example in the treatment of skin conditions (e.g. sunburn, burns, eczema, dermatitis, psoriasis); ophthalmic diseases; lung disorders (e.g. asthma, bronchitis, emphysema, allergic rhinitis, non-allergic rhinitis, cough, respiratory distress syndrome, pigeon fancier's disease, farmer's lung, chronic obstructive pulmonary disease, (COPD); gastrointestinal tract disorders (e.g. Crohn's disease, ulcerative colitis, coeliac disease, regional ileitis, irritable bowel syndrome, inflammatory bowel disease, gastroesophageal reflux disease); other conditions with an inflammatory component such as migraine, multiple sclerosis, myocardial ischemic.

In one embodiment, the compounds of the invention are useful in the treatment of neuropathic pain or inflammatory pain as described herein.

Without wishing to be bound by theory, other diseases or conditions that may be mediated by modulation of voltage-gated sodium channels are selected from the list consisting of [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):
ii) Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).
iii) Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):
iv) Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:
v) Enhancement of cognition including the treatment of cognition impairment in other diseases such as schizophrenia, bipolar disorder, depression, other psychiatric disorders and psychotic conditions associated with cognitive impairment, e.g. Alzheimer's disease:
vi) Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome:
vi) Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):
vii) Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).
viii) Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):
ix) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9): and
x) Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).
xi) Impulse control disorder” including: Intermittent Explosive Disorder (312.34), Kleptomania (312.32), Pathological Gambling (312.31), Pyromania (312.33), Trichotillomania (312.39), Impulse-Control Disorders Not Otherwise Specified (312.3), Binge Eating, Compulsive Buying, Compulsive Sexual Behaviour and Compulsive Hoarding.

In another embodiment, diseases or conditions that may be mediated by modulation of voltage gated sodium channels are depression or mood disorders

In another embodiment, diseases or conditions that may be mediated by modulation of voltage gated sodium channels are substance related disorders.

In a further embodiment, diseases or conditions that may be mediated by modulation of voltage gated sodium channels are Bipolar Disorders (including Bipolar I Disorder, Bipolar II Disorder (i.e. Recurrent Major Depressive Episodes with Hypomanic Episodes) (296.89), Cyclothymic Disorder (301.13) or Bipolar Disorder Not Otherwise Specified (296.80)).

In a still further embodiment, diseases or conditions that may be mediated by modulation of voltage gated sodium channels are Nicotine-Related Disorders such as Nicotine Dependence (305.1), Nicotine Withdrawal (292.0) or Nicotine-Related Disorder Not Otherwise Specified (292.9).

Compounds of the invention may also be useful in the treatment and/or prevention of disorders treatable and/or preventable with anti-convulsive agents, such as epilepsy including post-traumatic epilepsy, obsessive compulsive disorders (OCD), sleep disorders (including circadian rhythm disorders, insomnia & narcolepsy), tics (e.g. Giles de la Tourette's syndrome), ataxias, muscular rigidity (spasticity), and temporomandibular joint dysfunction.

Compounds of the invention may also be useful in the treatment of bladder hyperrelexia following bladder inflammation.

Compounds of the invention may also be useful in the treatment of neurodegenerative diseases and neurodegeneration such as dementia, particularly degenerative dementia (including senile dementia, Alzheimer's disease, Pick's disease, Huntington's chorea, Parkinson's disease and Creutzfeldt-Jakob disease, motor neuron disease); The compounds may also be useful for the treatment of amyotrophic lateral sclerosis (ALS) and neuroinflamation.

Compounds of the invention may also be useful in neuroprotection and in the treatment of neurodegeneration following stroke, cardiac arrest, pulmonary bypass, traumatic brain injury, spinal cord injury or the like.

Compounds of the invention may also be useful in the treatment of tinnitus, and as local anaesthetics.

Compounds of the invention may also be used in combination with other therapeutic agents. The invention thus provides, in a further embodiment, a combination comprising the crystalline form as defined herein together with a further therapeutic agent for use in the treatment of diseases and conditions mediated by modulation of voltage-gated sodium channels, such as pain.

Compounds of the invention may be used in combination with other medicaments indicated to be useful in the treatment or prophylaxis of pain (i.e. analgesics). Such therapeutic agents include for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO 99/012930); 5-lipoxygenase inhibitors; NSAIDs (non-steroidal anti-inflammatory drugs) such as diclofenac, indomethacin, nabumetone or ibuprofen; bisphosphonates, leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists or memantine; ligands for the α2δ-subunit of voltage gated calcium channels, such as gabapentin, pregabalin and solzira; tricyclic antidepressants such as amitriptyline; neurone stabilising antiepileptic drugs; cholinesterase inhibitors such as galantamine; mono-aminergic uptake inhibitors such as venlafaxine; opioid analgesics; local anaesthetics; 5HT1 agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP4 receptor ligands; EP₂ receptor ligands; EP₃ receptor ligands; EP₄ agonists and EP₂ agonists; EP₄ antagonists; EP₂ antagonists and EPs antagonists; cannabinoid receptor ligands; bradykinin receptor ligands; vanilloid receptor or Transient Receptor Potential (TRP) ligands; and purinergic receptor ligands, including antagonists at P2X3, P2X2/3, P2X4, P2X7 or P2X4/7; KCNQ/Kv7 channel openers, such as retigabine; additional COX-2 inhibitors are disclosed in U.S. Pat. Nos. 5,474,995, 5,633,272, 5,466,823, 6,310,099 and 6,291,523; and in WO 96/25405, WO 97/38986, WO 98/03484, WO 97/14691, WO 99/12930, WO 00/26216, WO 00/52008, WO 00/38311, WO 01/58881 and WO 02/18374.

In one embodiment, the present invention is directed to co-therapy, adjunctive therapy or combination therapy, comprising administration of the compounds of the invention and one or more analgesics (e.g. tramadol or amitriptyline), anticonvulsant drugs (e.g. gabapentin, neurontin or pregabalin Lyrica)) or antidepressant drugs (e.g. duloxetine (i.e. Cymbalta) or venlafaxine).

In this embodiment, therapeutically effective amount shall mean that amount of the combination of agents taken together so that the combined effect elicits the desired biological or medicinal response. For example, the therapeutically effective amount of co-therapy comprising administration of the compound of the invention and at least one suitable analgesic, anticonvulsant or antidepressant drugs would be the amount of a compound of the invention and the amount of the suitable analgesic, anticonvulsant or antidepressant drugs that when taken together or sequentially have a combined effect that is therapeutically effective. Further, it will be recognized by one skilled in the art that in the case of co-therapy with a therapeutically effective amount, the amount of a compound of the invention and/or the amount of the suitable analgesic, anticonvulsant or antidepressant drugs individually may or may not be therapeutically effective.

As used herein, the terms “co-therapy”, “adjunctive therapy” and “combination therapy” shall mean treatment of a subject in need thereof by administering one or more analgesic, anticonvulsant or antidepressant agent(s) and a compound of the invention, wherein the compound of the invention and the analgesic, anticonvulsant or antidepressant agent(s) are administered by any suitable means, simultaneously, sequentially, separately or in a single pharmaceutical formulation.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

Where the compound of the invention and the analgesic, anticonvulsant or antidepressant agent(s) are administered in separate dosage forms, the number of dosages administered per day for each compound may be the same or different. The compound of the invention and the analgesic, anticonvulsant or antidepressant agent(s) may be administered via the same or different routes of administration. Examples of suitable methods of administration include, but are not limited to, oral, intravenous (iv), intramuscular (im), subcutaneous (sc), intranasal, transdermal, and rectal. Compounds may also be administered directly to the nervous system including, but not limited to, intracerebral, intraventricular, intracerebroventhcular, intrathecal, intracisternal, intraspinal and/or peri-spinal routes of administration by delivery via intracranial or intravertebral needles and/or catheters with or without pump devices. The compound of the invention and the analgesic, anticonvulsant or antidepressant agent(s) may be administered according to simultaneous or alternating regimens, at the same or different times during the course of the therapy, concurrently in divided or single forms.

Advantageously, the compound of the invention may be administered in a single daily dose, or the total daily dosage may be administered in divided doses of two, three or four times daily.

According to a further embodiment of the invention, there is provided the crystalline form as defined herein for use in therapy.

According to a further embodiment of the invention, there is provided the crystalline form as defined herein for use in the treatment of a disease or condition mediated by modulation of voltage-gated sodium channels.

According to a further embodiment of the invention, there is provided the use of the crystalline form as defined herein in the manufacture of a medicament for the treatment of a disease or condition mediated by modulation of voltage-gated sodium channels.

According to a further embodiment of the invention, there is provided a method of treating a disease or condition mediated by modulation of voltage-gated sodium channels which comprises administering a therapeutically effective amount of the crystalline form as defined herein to a subject in need thereof.

The term “subject” as used herein, refers to an animal, preferably a mammal, most preferably a human adult, child or infant, who has been the object of treatment, observation or experiment.

It will be appreciated that references herein to “treatment” extend to prophylaxis, prevention of recurrence and suppression or amelioration of symptoms (whether mild, moderate or severe) as well as the treatment of established conditions.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of one or more of the symptoms of the disease or disorder being treated; and/or reduction of the severity of one or more of the symptoms of the disease or disorder being treated.

The compound of the invention may be administered as the raw chemical but the active ingredient is preferably presented as a pharmaceutical composition.

Thus, according to a further embodiment of the invention, there is provided a pharmaceutical composition comprising the crystalline form as defined herein with one or more pharmaceutically acceptable carrier(s), diluents(s) and/or excipient(s).

As used herein, the term “composition” is intended to encompass a product comprising the specified ingredients in the specified amounts, as well as any product which results, directly or indirectly, from combinations of the specified ingredients in the specified amounts.

Since the compounds described herein are intended for use in pharmaceutical compositions it will readily be understood that they are each preferably provided in substantially pure form, for example at least 60% pure, more suitably at least 75% pure and preferably at least 85%, especially at least 98% pure (% are given on a weight for weight basis). Impure preparations of the compounds may be used for preparing the more pure forms used in the pharmaceutical compositions.

According to a further embodiment of the invention, there is provided a pharmaceutical composition comprising a compound of the invention for use in the treatment of a disease or condition mediated by modulation of voltage-gated sodium channels.

In one embodiment, the pharmaceutical composition comprises one or more pharmaceutically acceptable carrier(s), diluent(s) and/or excipient(s). The carrier, diluent and/or excipient must be “acceptable” in the sense of being compatible with the other ingredients of the composition and not deleterious to the recipient thereof.

Pharmaceutical compositions containing the compound of the invention as the active ingredient can be prepared by intimately mixing the compound with a pharmaceutical carrier according to conventional pharmaceutical compounding techniques. These procedures may involve mixing, granulating and compressing or dissolving the ingredients as appropriate to the desired preparation.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The compounds or their pharmaceutically acceptable salts may be administered by any convenient method, e.g. by oral, parenteral, buccal, sublingual, nasal, rectal or transdermal administration, and the pharmaceutical compositions adapted accordingly, for administration to mammals including humans.

The compounds or their pharmaceutically acceptable salts which are active when given orally can be formulated as liquids or solids, e.g. as syrups, suspensions, emulsions, tablets, capsules or lozenges.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non aqueous vehicles (which may include edible oils), for example almond oil, oily esters such as glycerine, propylene glycol, or ethyl alcohol; preservatives, for example methyl or propyl p hydroxybenzoate or sorbic acid, and, if desired, conventional flavouring or colouring agents.

Typical parenteral compositions consist of a solution or suspension of the active ingredient in a sterile vehicle, water being preferred, or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration. The compound, depending on the vehicle and concentration used, can be either suspended or dissolved in the vehicle. In preparing solutions the compound can be dissolved in water for injection and filter-sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluoro-chloro-hydro-carbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin. Compositions for rectal administration are conveniently in the form of suppositories containing a conventional suppository base such as cocoa butter. Compositions suitable for transdermal administration include ointments, gels and patches.

In one embodiment the composition is in unit dose form such as a tablet, capsule or ampoule.

The dose of the compound or a pharmaceutically acceptable salt thereof, used in the treatment of the abovementioned disorders or diseases will vary in the usual way with the particular disorder or disease being treated, the weight of the subject and other similar factors. However, as a general rule, suitable unit doses may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be in the range of 50 mg to 1500 mg per day, for example 120 mg to 1000 mg per day. Such therapy may extend for a number of weeks or months.

It will be recognised by one of skill in the art that the optimal quantity and spacing of individual dosages of the compound of the invention will be determined by the nature and extent of the condition being treated, the form, route and site of administration, and the particular mammal being treated, and that such optimums can be determined by conventional techniques. In addition, factors associated with the particular patient being treated, including patient age, weight, diet and time of administration, will result in the need to adjust dosages. It will also be appreciated by one of skill in the art that the optimal course of treatment, i.e., the number of doses of a compound of the invention given per day for a defined number of days, can be ascertained by those skilled in the art using conventional course of treatment determination tests.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

### EXAMPLES

The invention is illustrated by the Examples described below:

### Methods

Single crystal analyses were performed either using a Bruker APEX-II CCD diffractometer (173K) or a Bruker D8 Quest diffractometer (293K). Samples were mounted on a nylon loop with paratone oil for data collection using a MoKα radiation source.

All pXRD spectra were obtained using one of the three following methods:

| | Method 1 | Method 2 | Method 3 |
|---|---|---|---|
| Manufacturer | Bruker | Bruker | PANalytical |
| Model | D8 Advance | D8 Advance | Empyrean |
| Detector | PSD Lynx | PSD Lynx | X'Celerator |
| Radiation Source | Cu | Cu | Cu |
| Generator Voltage (kV) | 40 | 40 | 45 |
| Generator Current (mA) | 40 | 40 | 40 |
| Start 2Θ Angle (°) | 3.0 | 3.0 | 3.0 |
| End 2 Θ Angle (°) | 39.8883 | 38.9681 | 40.0 |
| Step Size (°) | 0.0164 | 0.0164 | 0.0167 |
| Scan Step Time (s) | 0.1 | 0.5 | 17.8 |
| Forms | 2, 3 | 1, 4-8 | 9, 10 |

All values for peaks provided herein are intended to refer to the value in 2θ Diffraction (°) with a margin of error selected from: ±0.5, such as ±0.25, in particular ±0.15, especially ±0.1, more especially ±0.05, most especially ±0.01.

Using Olex2 (Dolomanov et al. (2009) J. Appl. Cryst. 42, 339-341), the structure was solved with the SheIXS (Sheldrick (2008) Acta Crystallogr A, 64(1), 112-122) structure solution program, using the Direct Methods solution method. The model was refined with version 2014/6 of XL (Sheldrick, 2008) using Least Squares minimization.

### Example 1: (5R)-5-(4-{[(2-Fluorophenyl)methyl]oxy}phenyl)-L-prolinamide hydrochloride (E1)

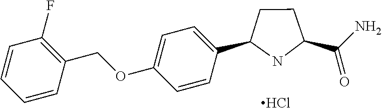 [SMILES: NC(=O)[C@@H]1CC[C@H](c2ccc(OCc3ccccc3F)cc2)N1 ; Cl]

The compound of Example 1 may be prepared as described in Example 2, Procedures 1 to 5 of WO 2007/042239.

### Example 2: (5R)-5-(4-{[(2-Fluorophenyl)methyl]oxy}phenyl)-L-prolinamide hydrochloride Form 1 (Anhydrous A) (E2)

25.0 mg of Example 1 was added to a 3 mL scintillation vial. THF (2.00 mL) was added and the resulting suspension stirred for 10 minutes. The suspension was filtered through a 0.45 μm PTFE filter and the filtrate vial placed inside a 20 mL scintillation vial. Hexanes (2 mL) were placed in the outer vial, the entire system sealed and stored at room temperature for 3 days, after which time a crop of colorless crystals was evident in the 3 mL vial. One of these crystals was selected for a single crystal X-ray diffraction experiment. Full characterisation is shown in FIGS. 1 and 2 and Tables 1 and 2 below.

TABLE 1
Single Crystal Structural Information
and Refinement Parameters for Form 1.

| Parameter | Form 1 (Anhydrous A) | |
|---|---|---|
| Empirical formula | C₁₈H₂₀N₂O₂FCl₃ | |
| M/g · mol⁻¹ | 350.81 | |
| T/K | 173 | (2) |
| Color | Colorless | |
| Crystal system | Monoclinic | |
| Space group | P2₁ | |
| a/Å | 5.5108 | (12) |
| b/Å | 8.5085 | (18) |
| c/Å | 36.887 | (8) |
| β/° | 91.218 | (3) |
| V/Å³ | 1729.2 | (6) |

| | |
|---|---|
| Z | 4 |
| Dc/g cm⁻³ | 1.348 |
| μ/mm⁻¹ | 0.244 |
| Crystal size/mm | 0.16 × 0.14 × 0.05 |
| Reflections collected | 14384 |
| R(int) | 0.0431 |
| Data/restraints/parameters | 4889/1/465 |
| R1 [I > 2 (I)] | 0.0443 |
| wR2 (all data) | 0.1025 |
| Largest peak, hole/e Å⁻³ | 0.384, −0.187 |

TABLE 2
List of pXRD diffraction peaks for Form 1 extrapolated
from FIG. 2. Peaks in bold represent the strongest
diffraction peaks based on the calculated pattern).

| Form | 2θ Diffraction (°) |
|---|---|
| Form 1 | 9.56, 11.48, 12.71, 14.30, 16.23, 17.49, 17.87, 19.23, |
| (Anhydrous A) | 19.74, 19.87, 20.40, 21.09, 21.47, 22.47, 23.06, 23.87, |
| | 24.10, 26.61, 26.79, 27.37, 28.09, 31.89, 32.66, 33.25, |
| | 34.20 |

### Example 3: (5R)-5-(4-{[(2-Fluorophenyl)methyl]oxy}phenyl)-L-prolinamide hydrochloride Form 2 (Ethanol) (E3)

25.0 mg of Example 1 was added to a 3 mL scintillation vial. EtOH (1.00 mL) was added and the resulting suspension stirred for 10 minutes. The suspension was filtered through a 0.45 μm PTFE filter and hexanes (0.8 mL) added to the filtrate. The vial was closed and left undisturbed for 2 days, over which time a crop of colorless crystals was obtained. One of these crystals was isolated and subjected to analysis by single crystal X-ray diffraction. Full characterisation is shown in FIGS. 3 and 4 and Tables 3 and 4 below.

TABLE 3
Single Crystal Structural Information
and Refinement Parameters for Form 2.

| Parameter | Form 2 (Ethanol) | |
|---|---|---|
| Empirical formula | C₁₈H₂₀N₂O₂FCl₃•(C₂H₆O) | |
| M/g · mol⁻¹ | 396.88 | |
| T/K | 173 | (2) |
| Color | Colorless | |
| Crystal system | Monoclinic | |
| Space group | P2₁ | |
| a/Å | 5.74500 | (10) |
| b/Å | 8.39580 | (10) |
| c/Å | 21.2276 | (3) |
| β/° | 93.7050 | (10) |
| V/Å³ | 1021.75 | (3) |

| | |
|---|---|
| Z | 2 |
| Dc/g cm⁻³ | 1.290 |
| μ/mm⁻¹ | 1.920 |
| Crystal size/mm | 0.36 × 0.27 × 0.02 |
| Reflections collected | 13192 |
| R(int) | 0.0277 |
| Data/restraints/parameters | 3801/1/248 |
| R1 [I > 2 (I)] | 0.0298 |
| wR2 (all data) | 0.0806 |
| Largest peak, hole/e Å⁻³ | 0.423, −0.183 |

TABLE 4
List of pXRD diffraction peaks for Form 2 extrapolated from
FIG. 4. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 2 | 4.16, 8.31, 11.29, 12.45, 13.36, 15.43, 15.69, 16.24, |
| (Ethanol) | 18.67, 18.92, 20.03, 20.49, 21.04, 21.45, 22.05, 22.61, |
| | 23.07, 23.57, 24.48, 26.30, 27.16, 28.57 |

### Example 4: (5R)-5-(4-{[(2-Fluorophenyl)methyl]oxy}phenyl)-L-prolinamide hydrochloride Form 3 (Methanol) (E4)

100.1 mg Example 1 was added to a 3 mL scintillation vial. MeOH (1.00 mL) was added and the resulting suspension stirred for 10 minutes. The suspension was filtered through a 0.45 μm PTFE filter and the filtrate vial closed and left undisturbed for 10 minutes, after which time a crop of colorless crystals was present on the vial bottom. A single crystal from this crop was analyzed by single crystal X-ray diffraction for structural elucidation. Full characterisation is shown in FIGS. 5 and 6 and Tables 5 and 6 below.

TABLE 5
Single Crystal Structural Information
and Refinement Parameters for Form 3.

| Parameter | Form 3 (Methanol) | |
|---|---|---|
| Empirical formula | C₁₈H₂₀N₂O₂FCl₃•(C_{1.5}H₆O_{1.5}) | |
| M/g mol⁻¹ | 398.83 | |
| T/K | 173 | (2) |
| Color | Colorless | |
| Crystal system | Monoclinic | |
| Space group | C2 | |
| a/Å | 31.14 | (9) |
| b/Å | 5.6871 | (2) |
| c/Å | 11.8317 | (4) |
| β/° | 90.3955 | (17) |
| V/Å³ | 2086.83 | (12) |

| | |
|---|---|
| Z | 4 |
| Dc/g cm⁻³ | 1.256 |
| μ/mm⁻¹ | 1.886 |
| Crystal size/mm | 0.24 × 0.15 × 0.08 |
| Reflections collected | 14545 |
| R(int) | 0.0267 |
| Data/restraints/parameters | 3881/2/258 |
| R1 [I > 2 (I)] | 0.0477 |
| wR2 (all data) | 0.1322 |
| Largest peak, hole/e Å⁻³ | 0.880, −1.097 |

TABLE 6
List of pXRD diffraction peaks for Form 3 extrapolated from
FIG. 6. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 3 | 7.55, 9.53, 14.98, 16.05, 17.70, 18.85, 19.30, 21.94, |
| (methanol) | 22.45, 22.79, 23.30, 24.18, 25.23, 26.07, 26.60, 27.61, |
| | 28.76, 29.62, 31.00, 32.20, 32.91 |

### Example 5: (5R)-5-(4-{[(2-Fluorophenyl)methyl]oxy}phenyl)-L-prolinamide hydrochloride Form 4 (1-Propanol) (E5)

25.0 mg of Example 1 was added to a 3 mL scintillation vial. 1-propanol (7.00 mL) was added and the resulting suspension stirred for 10 minutes. The suspension was filtered through a medium glass frit to create a saturated 1-propanol solution. 1 mL of this solution was added to a 20 mL scintillation vial and hexanes (9 mL) added to the vial. The vial was closed and left undisturbed for 2 days, over which time a crop of colorless crystals was obtained. One of these crystals was analyzed by single crystal X-ray diffraction. Full characterisation is shown in FIGS. 7 and 8 and Tables 7 and 8 below.

TABLE 7
Single Crystal Structural Information
and Refinement Parameters for Form 4.

| Parameter | Form 4 (1-Propanol) | |
|---|---|---|
| Empirical formula | C₁₈H₂₀N₂O₂FCl₃•(C₃H₈O) | |
| M/g · mol⁻¹ | 410.90 | |
| T/K | 173 | (2) |
| Color | Colorless | |
| Crystal system | Monoclinic | |
| Space group | P2₁ | |
| a/Å | 5.70510 | (10) |
| b/Å | 8.22950 | (10) |
| c/Å | 22.4759 | (3) |
| β/° | 93.7870 | (10) |
| V/Å³ | 1052.94 | (3) |

| | |
|---|---|
| Z | 2 |
| Dc/g cm⁻³ | 1.296 |
| μ/mm⁻¹ | 1.881 |
| Crystal size/mm | 0.31 × 0.15 × 0.04 |
| Reflections collected | 14050 |
| R(int) | 0.0473 |
| Data/restraints/parameters | 3636/1/255 |
| R1 [I > 2 (I)] | 0.0330 |
| wR2 (all data) | 0.0814 |
| Largest peak, hole/e Å⁻³ | 0.221, −0.169 |

TABLE 8
List of pXRD diffraction peaks for Form 4 extrapolated from
FIG. 8. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 4 | 3.92, 7.85, 11.37, 11.78, 15.82, 16.94, 18.92, 20.91, |
| (1-Propanol) | 21.72, 22.97, 23.77, 24.47, 25.46, 26.17, 28.15, 31.66, |
| | 34.84 |

### Example 6: (5R)-5-(4-{[(2-Fluorophenyl)methyl]oxy}phenyl)-L-prolinamide hydrochloride Form 5 (1-Butanol) (E6)

50.8 mg of Example 1 was added to a 20 mL scintillation vial. 1-Butanol (4.00 mL) was added and the resulting suspension stirred for 10 minutes. The suspension was heated to 100° C. and continued to stir for 10 minutes, at which point a clear solution was obtained. Example 1 was added in small increments to the stirred solution until a suspension was obtained. At this point 500 μL 1-butanol was added and the resulting clear solution allowed to stir for 5 minutes. The solution was filtered hot through a pre-heated 0.45 μm PTFE filter and syringe. The vial was closed and the temperature reduced to 70° C. and the vial left undisturbed overnight. The next day a large crop of colorless crystals was evident. A saturated suspension of Example 1 in 1-butanol was made at room temperature by stirring 5.5 mg Example 1 in 1 mL 1-butanol. The suspension was filtered through a 0.45 μm PTFE filter. The crystal-containing solution (still at 70° C.) was decanted and the room temperature saturated solution of Example 1 in 1-butanol was added. After allowing the entire system to cool to equilibrate at room temperature, a few crystals were selected and transferred along with mother liquor to a 3 mL vial. This sample was sent for single crystal analysis and the structure of the 1-butanol solvate obtained. Full characterisation is shown in FIGS. 9 and 10 and Tables 9 and 10 below.

TABLE 9
Single Crystal Structural Information
and Refinement Parameters for Form 5.

| Parameter | Form 5 (1-Butanol) | |
|---|---|---|
| Empirical formula | C₁₈H₂₀N₂O₂FCl₃•(C₄H₁₀O) | |
| M/g · mol⁻¹ | 424.93 | |
| T/K | 173 | (2) |
| Color | Colorless | |
| Crystal system | Monoclinic | |
| Space group | P2₁ | |
| a/Å | 5.72350 | (10) |
| b/Å | 8.23470 | (10) |
| c/Å | 23.3724 | (3) |
| β/° | 96.4520 | (10) |
| V/Å³ | 1094.59 | (3) |

| | |
|---|---|
| Z | 2 |
| Dc/g cm⁻³ | 1.289 |
| μ/mm⁻¹ | 1.826 |
| Crystal size/mm | 0.48 × 0.22 × 0.08 |
| Reflections collected | 20526 |
| R(int) | 0.0303 |
| Data/restraints/parameters | 4015/1/281 |
| R1 [I > 2 (I)] | 0.0252 |
| wR2 (all data) | 0.0645 |
| Largest peak, hole/e Å⁻³ | 0.166, −0.181 |

TABLE 10
List of pXRD diffraction peaks for Form 5 extrapolated from
FIG. 10. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 5 | 3.92, 7.78, 11.45, 15.57, 15.72, 16.56, 18.95, 19.74, |
| (1-Butanol) | 21.24, 21.53, 21.88, 23.14, 24.43, 25.54, 26.35, 27.20, |
| | 28.32, 31.74, 33.37, 34.66 |

### Example 7: (5R)-5-(4-{[(2-Fluorophenyl)methyl]oxy}phenyl)-L-prolinamide hydrochloride Form 6 (2-Methoxyethanol) (E7)

24.3 mg Example 1 was added to a 3 mL vial and suspended in 3 mL 2-methoxyethanol. The suspension was stirred for 15 min at room temperature and subsequently filtered through a medium glass frit resulting in a 2-methoxyethanol saturated solution. 1 mL of the saturated solution was added to a 3 mL vial, 500 μL hexanes added and the vial sealed and stored at ambient conditions for 2 days. A small crop of crystals was obtained and sent for single crystal XRD analysis. Full characterisation is shown in FIGS. 11 and 12 and Tables 11 and 12.

TABLE 11
Single Crystal Structural Information
and Refinement Parameters for Form 6.

| Parameter | Form 6 (2-Methoxyethanol) | |
|---|---|---|
| Empirical formula | C₁₈H₂₀N₂O₂FCl₃•(C₃H₈O₂) | |
| M/g · mol⁻¹ | 426.90 | |
| T/K | 173 | (2) |
| Color | Colorless | |
| Crystal system | Monoclinic | |
| Space group | P2₁ | |
| a/Å | 5.67000 | (10) |
| b/Å | 8.21240 | (10) |
| c/Å | 23.0824 | (4) |
| β/° | 95.2170 | (10) |
| V/Å³ | 1070.36 | (3) |

| | |
|---|---|
| Z | 2 |
| Dc/g cm⁻³ | 1.325 |
| μ/mm⁻¹ | 1.907 |
| Crystal size/mm | 0.34 × 0.07 × 0.04 |
| Reflections collected | 11528 |
| R(int) | 0.0461 |
| Data/restraints/parameters | 3713/4/272 |
| R1 [I > 2 (I)] | 0.0402 |
| wR2 (all data) | 0.0934 |
| Largest peak, hole/e Å⁻³ | 0.459, −0.194 |

TABLE 12
List of pXRD diffraction peaks for Form 6 extrapolated from
FIG. 12. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 6 (2- | 3.86, 7.70, 11.54, 15.38, 19.05, 19.30, 19.96, 21.56, |
| Methoxyethanol) | 21.90, 23.17, 24.51, 25.53, 31.79 |

### Example 8: (5R)-5-(4-{[(2-Fluorophenyl)methyl]oxy}phenyl)-L-prolinamide hydrochloride Form 7 (Ethylene Glycol) (E8)

48.9 mg Example 1 was added to a 20 mL scintillation vial. 4 mL ethylene glycol was added and the suspension heated until fully dissolved (70° C.). The solution was slowly cooled (˜5° C./30 min) resulting in a crop of single crystals. A single crystal was isolated and sent in mother liquor for analysis which established the identity as the solvate of the title compound. Full characterisation is shown in FIGS. 13 and 14 and Tables 13 and 14 below.

TABLE 13
Single Crystal Structural Information
and Refinement Parameters for Form 7.

| Parameter | Form 7 (Ethylene Glycol) | |
|---|---|---|
| Empirical formula | C₁₈H₂₀N₂O₂FCl₃•(C₂H₆O₂) | |
| M/g · mol⁻¹ | 412.88 | |
| T/K | 173 | (2) |
| Color | Colorless | |
| Crystal system | Monoclinic | |
| Space group | P2₁ | |
| a/Å | 5.72400 | (14) |
| b/Å | 8.561 | (2) |
| c/Å | 21.123 | (5) |
| β/° | 94.383 | (3) |
| V/Å³ | 1032.0 | (4) |

| | |
|---|---|
| Z | 2 |
| Dc/g cm⁻³ | 1.329 |
| μ/mm⁻¹ | 0.222 |
| Crystal size/mm | 0.29 × 0.10 × 0.08 |
| Reflections collected | 10161 |
| R(int) | 0.0317 |
| Data/restraints/parameters | 3422/1/290 |
| R1 [I > 2 (I)] | 0.0393 |
| wR2 (all data) | 0.0819 |
| Largest peak, hole/e Å⁻³ | 0.264, −0.169 |

TABLE 14
List of pXRD diffraction peaks for Form 7 extrapolated from
FIG. 14. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 7 | 8.38, 11.29, 12.69, 13.40, 15.54, 15.89, 16.40, 18.74, |
| (Ethylene | 18.95, 19.79, 20.12, 20.73, 21.24, 21.90, 22.43, 23.26, |
| Glycol) | 23.78, 24.43, 26.35, 26.02, 27.06, 27.71, 28.50, 29.47, |
| | 29.68, 30.51, 30.66, 32.96, 33.57, 33.89, 35.75, 37.86 |

### Example 9: (5R)-5-(4-{[(2-Fluorophenyl)methyl]oxy}phenyl)-L-prolinamide hydrochloride Form 8 (Propylene Glycol) (E9)

74.1 mg Example 1 was suspended in 1 mL propylene glycol, stirred for 10 min and filtered through a medium glass frit to create a saturated solution. 100 μL saturated solution added to a 3 mL vial. Added 900 μL ethyl acetate and the vial sealed and stored overnight at ambient conditions. The next day a crop of crystals was evident. These crystals were sent in mother liquor for single crystal analysis and used to establish the identity as the solvate of the title compound. Full characterisation is shown in FIGS. 15 and 16 and Tables 15 and 16 below.

TABLE 15
Single Crystal Structural Information
and Refinement Parameters for Form 8.

| Parameter | Form 8 (Propylene Glycol) | |
|---|---|---|
| Empirical formula | C₁₈H₂₀N₂O₂FCl₃•(C₃H₈O₂) | |
| M/g · mol⁻¹ | 426.90 | |
| T/K | 173 | (2) |
| Color | Colorless | |
| Crystal system | Monoclinic | |
| Space group | C2 | |
| a/Å | 16.2771 | (3) |
| b/Å | 5.56450 | (10) |
| c/Å | 24.5949 | (5) |
| β/° | 104.8505 | (14) |
| V/Å³ | 2153.25 | (7) |

| | |
|---|---|
| Z | 4 |
| Dc/g cm⁻³ | 1.317 |
| μ/mm⁻¹ | 1.896 |
| Crystal size/mm | 0.39 × 0.09 × 0.07 |
| Reflections collected | 13264 |
| R(int) | 0.0739 |
| Data/restraints/parameters | 3319/73/352 |
| R1 [I > 2 (I)] | 0.0725 |
| wR2 (all data) | 0.1547 |
| Largest peak, hole/e Å⁻³ | 0.770, −0.255 |

TABLE 16
List of pXRD diffraction peaks for Form 8 extrapolated from
FIG. 16. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 8 | 7.47, 10.86, 11.21, 11.85, 13.80, 14.95, 16.42, 16.86, |
| (Propylene | 17.59, 18.71, 21.80, 22.48, 25.22, 25.46, 27.06, |
| Glycol) | |

### Example 10: (5R)-5-(4-{[(2-Fluorophenyl)methyl]oxy}phenyl)-L-prolinamide hydrochloride Form 9 (Anhydrous B) (E10)

10 mg of Example 1 was dissolved in 0.5-1.5 mL of acetonitrile in a 1.5-mL glass vial, equilibrated at 50° C. for an hour. The visually clear solutions were filtered using a nylon membrane (pore size of 0.45 μm) and then subjected to evaporation at 50° C. after vials were sealed using Parafilm® with some pinholes. The obtained solid was isolated for single crystal analysis. Full characterisation is shown in FIGS. 17 and 18 and Tables 17 and 18 below.

TABLE 17
Single Crystal Structural Information
and Refinement Parameters for Form 9.

| Parameter | Form 9 (Anhydrous B) | |
|---|---|---|
| Empirical formula | C₁₈H₂₀N₂O₂FCl₃ | |
| M/g · mol⁻¹ | 350.81 | |
| T/K | 293 | (2) |
| Color | Colorless | |
| Crystal system | Monoclinic | |
| Space group | P2₁ | |
| a/Å | 7.1929 | (12) |
| b/Å | 8.9436 | (13) |
| c/Å | 14.021 | (2) |
| β/° | 101.570 | (5) |
| V/Å³ | 883.7 | (2) |

| | |
|---|---|
| Z | 2 |
| Dc/g cm⁻³ | 1.318 |
| μ/mm⁻¹ | 0.238 |
| Crystal size/mm | 0.50 × 0.20 × 0.04 |
| Reflections collected | 14322 |
| R(int) | 0.0257 |
| Data/restraints/parameters | 3454/1/217 |
| R1 [I > 2 (I)] | 0.0350 |
| wR2 (all data) | 0.0811 |
| Largest peak, hole/e Å⁻³ | 0.21, −0.20 |

TABLE 18
List of pXRD diffraction peaks for Form 9 extrapolated from
FIG. 18. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 9 | 6.52, 12.95, 16.33, 19.44, 19.85, 21.86, 22.23, 23.56, |
| (Anhydrous B) | 25.27, 26.51, 27.21, 27.86 |

### Example 11: (5R)-5-(4-{[(2-Fluorophenyl)methyl]oxy}phenyl)-L-prolinamide hydrochloride Form 10 (Anhydrous C) (E11)

A saturated solution of Example 1 in methanol was prepared and centrifuged. 100 μL of the resulting solution was added into a 3 mL vial containing 2 mL deionized water. The resulting clear solution was evaporated at room temperature resulting in a crop of crystals suitable for X-ray diffraction. Full characterisation is shown in FIGS. 19 and 20 and Tables 19 and 20 below.

TABLE 19
Single Crystal Structural Information
and Refinement Parameters for Form 10.

| Parameter | Form 10 (Anhydrous C) | |
|---|---|---|
| Empirical formula | C₁₈H₂₀N₂O₂FCl₃ | |
| M/g · mol⁻¹ | 350.81 | |
| T/K | 293 | (2) |
| Color | Colorless | |
| Crystal system | Monoclinic | |
| Space group | P2₁ | |
| a/Å | 7.1352 | (10) |
| b/Å | 5.8909 | (7) |
| c/Å | 19.540 | (3) |
| β/° | 92.493 | (4) |
| V/Å³ | 820.56 | (19) |

| | |
|---|---|
| Z | 2 |
| Dc/g cm⁻³ | 1.420 |
| μ/mm⁻¹ | 0.257 |
| Crystal size/mm | 0.24 × 0.22 × 0.03 |
| Reflections collected | 17066 |
| R(int) | 0.0688 |
| Data/restraints/parameters | 3789/1/217 |
| R1 [I > 2 (I)] | 0.0411 |
| wR2 (all data) | 0.0841 |
| Largest peak, hole/e Å⁻³ | 0.38, −0.26 |

TABLE 20
List of pXRD diffraction peaks for Form 10 extrapolated
from FIG. 20. Peaks in bold represent the strongest diffraction
peaks based on the calculated pattern, underlined peaks
indicate a distinct diffraction peak with respect to Form
1 and bold and underlined peaks indicate both).

| Form | 2θ Diffraction (°) |
|---|---|
| Form 10 | 4.51, 8.99, 12.97, 17.48, 18.03, 19.45, 20.19, 21.39, |
| (Anhydrous C) | 21.76, 23.50, 25.34, 26.37, 27.19, 31.84, 33.14, 36.57 |

### Example 12: (5R)-5-(4-{[(2-Fluorophenyl)methyl]oxy}phenyl)-L-prolinamide hydrochloride (Anhydrous; Route D) (E12)

9.0 kg (2S,5R)-5-(4-((2-fluorobenzyl)oxy)phenyl)pyrrolidine-2-carboxamide (which may be prepared according to the route described in Description 6 of WO 2016/102967) in 127 kg ethanol was heated to a maximum of 50° C. to a complete dissolution. A 31 kg solution of 1.25M HCl in ethanol was added over 30 min. while maintaining the temperature within 20-25° C. The temperature of the reactor contents was maintained at 25-35° C., stirred for approximately 2 h, cooled to 0-5° C., and stirred for approximately 2 h. The slurry was filtered, and the wet cake washed with 2×14.0 kg cold (0-5° C.) ethanol. The product wet cake was dried under vacuum at temperatures 40-70° C. until no less than 0.4% moisture remained, to yield 9.6 kg dried product (E12).

### Example 13: (5R)-5-(4-{[(2-Fluorophenyl)methyl]oxy}phenyl)-L-prolinamide hydrochloride (Anhydrous; Route E) (E13)

A solution containing 60 kg (2S,5R)-5-(4-((2-fluorobenzyl)oxy)phenyl)pyrrolidine-2-carboxamide (which may be prepared according to the procedure described in Description 6 of WO 2016/102967) in 288 kg isopropanol was treated with 34.8 kg 20% aqueous hydrochloric acid solution while maintaining the temperature between 61-67° C. The mixture was cooled to 0-5° C. in approximately 4 h and stirred for approximately 2 h. The slurry was filtered and the product cake washed with 71 kg isopropanol. The wet cake was dried under vacuum at temperatures 40-70° C. until no greater than 0.4% moisture remained to yield 64 kg dried product (E13).

### Example 14: (5R)-5-(4-{[(2-Fluorophenyl)methyl]oxy}phenyl)-L-prolinamide hydrochloride (Anhydrous; Route F) (E14)

A solution of 118.4 kg (2S,5R)-5-(4-((2-fluorobenzyl)oxy)phenyl)pyrrolidine-2-carboxamide (which may be prepared according to the procedure described in Description 6 of WO 2016/102967) in 600.4 kg isopropanol, 36 kg water was treated with 6N hydrochloric acid solution in 20-40 min addition time while the temperature was maintained within 73-77° C. The mixture was cooled to 3-7° C. in 3 to 4 h and circulated through a IKA DR2000/10 high-shear mixer. The recirculation through the mill was terminated until particle size reduction was deemed complete as determined by in-process measurement of product particle size. The recirculation lines were rinsed with 90 kg isopropanol. The combined isopropanol and milled slurry mixture was heated to 58-62° C. in 3 h, held at 58-62° C. in 2 h, cooled to −2 to 2° C. in 3 h and held for 1-2 h at (−2)-2° C. The slurry was filtered and product cake washed with 92 kg isopropanol. The wet product was dried under vacuum at 45-80° C. until no greater than 0.1% isopropanol and no greater than 0.1% water was in the final dried product (E14; 133.3 kg).

### Powder Bulk Density Analysis

Solid forms prepared according to the procedures described for Routes D, E, and F (Examples 12-14, respectively) were subjected to powder bulk density analysis in accordance with the following procedure.

A ring shear tester (RST-XS.s, Dietmar Schulze, Wolfenbüttel, Germany) with RST CONTROL 95 software was used for the bulk density analysis. For this analysis, Low-Stress Shear Cell, XS-Lr0, bottom ring was over-filled with small portions of powder using a spatula. The excess powder was removed by gently scraping off the powder with the spatula until the powder was flush with the top of the ring. The bottom ring was weighed and the total mass was entered. When prompted the shear cell lid was attached to the loading rod and the filled bottom ring placed on the driving axle for the test. The initial bulk density was calculated by the control software from the mass of powder normalized by the volume of the cell. During the shear test, 5 preshear normal stresses, 0.1, 0.2, 0.3, 0.4, and 0.5 kPa were applied to the powder before it was sheared until steady state was achieved. At each preshear normal stress, the powder was sheared to failure at 5 increasing normal stresses between 0 and the preshear normal stress to generate corresponding shear stresses at failure. A yield locus was generated by applying a linear regression to the shear stresses as a function of the normal stress plot. The Major Principal Stress (x-axis) was the maximum value obtained when a Mohr's circle was drawn through the preshear normal stress and tangential to the yield locus. The volume of powder at each steady state was determined from the change in height of the lid and, together with the initial powder mass, used to calculate the powder bulk densities (y-axis).

The initial density results of this analysis are shown in Table 21. FIG. 21 shows the change in density at varying stress that are relevant for manufacturing purposes.

TABLE 21
Initial Density for Examples 12-14

| Product | Initial Density (g/cm³) |
|---|---|
| Route D; E12 | 0.254 |
| Route E; E13 | 0.565 |
| Route F; E14 | 0.512 |

Initial Density described in Table 21 corresponds to x = 0 in FIG. 21.

In general, the higher the density, the more ideal the initial packing state of the product. It can be seen from the results generated in Table 21 and FIG. 21 that the products of Route E (E13) and Route F (E14) demonstrated density results far superior to the product of Route D (E12) and therefore the Route E and F products may be better suited to a role as an active pharmaceutical ingredient.

In one embodiment, there is provided an anhydrous crystalline form of (5R)-5-(4-{[(2-fluorophenyl)methyl]oxy}phenyl)-L-prolinamide hydrochloride with an initial bulk density of at least about 0.4 g/cm³. In another embodiment, the anhydrous crystalline form has an initial bulk density of at least about 0.5 g/cm³. In another embodiment, the anhydrous crystalline form has an initial bulk density of at least about 0.6 g/cm³. In another embodiment, the anhydrgous crystalline form has an initial bulk density of about 0.4 g/cm³ to about 0.6 g/cm³. In another embodiment, the anhydrgous crystalline form has an initial bulk density of about 0.4 g/cm³ to about 0.5 g/cm³. In another embodiment, the anhydrgous crystalline form has an initial bulk density of about 0.5 g/cm³ to about 0.6 g/cm³.

### Powder Flow Function Analysis

Solid forms prepared according to the procedures described for Routes D, E, and F (Examples 12-14, respectively) were subjected to powder flow function analysis in accordance with the following procedure:

A ring shear tester (RST-XS.s, Dietmar Schulze, Wolfenbüttel, Germany) with RST CONTROL 95 software was used for the flow function analysis. For this analysis, Low-Stress Shear Cell, XS-Lr0, bottom ring was over-filled with small portions of powder using a spatula. The excess powder was removed by gently scraping off the powder with the spatula until the powder was flush with the top of the ring. The bottom ring was weighed and the total mass was entered. When prompted the shear cell lid was attached to the loading rod and the filled bottom ring placed on the driving axle for the test. To obtain a flow function, 5 yield loci were determined using the “Stress Walk” function in the control software. During the shear test, 5 preshear normal stresses, 0.1, 0.2, 0.3, 0.4, and 0.5 kPa were applied to the powder to generate the 5 yield loci. At each preshear normal stress, the powder was sheared to failure at 5 equally-spaced normal stresses between 0 and the preshear normal stress to generate corresponding shear stresses at failure. Each yield locus was derived by applying a linear regression to the shear stresses as a function of the normal stress plot. The Major Principal Stress was the maximum normal stress obtained when a Mohr's circle was drawn through the preshear normal stress and tangential to the yield locus. The unconfined yield strength was the maximum normal stress obtained when a second Mohr's circle was drawn tangential to the same yield locus but passing through origin. A plot of the unconfined yield strength as a function of the major principal stress is the flow function.

The results of this analysis are shown in FIG. 22 which demonstrates flow function curves. In general, a good powder flow is indicated by a lower flow function curve.

FIG. 22 demonstrates that the flow of Route E and F products (E13 and E14, respectively) is better (i.e. they have a lower flow function curve) than a product of Route D (E12). For smaller particle sizes, the flow of a Route F product (E14) may be lower than a Route E product (E13). It should be noted that the purpose of subjecting the products to major principal stress is to simulate the pharmaceutical manufacturing process.

In one embodiment, there is provided an anhydrous crystalline form of (5R)-5-(4-{[(2-fluorophenyl)methyl]oxy}phenyl)-L-prolinamide hydrochloride, characterised in that said anhydrous crystalline form has an unconfined yield strength of less than about 200 Pa at a major principal stress value of 500 Pa, tested in accordance with the powder flow function analysis herein. In another embodiment, the anhydrous crystalline form has an unconfined yield strength less than about 100 Pa at a major principal stress value of 500 Pa. In another embodiment, the anhydrous crystalline form has an unconfined yield strength from about 50 Pa to about 200 Pa at a major principal stress value of 500 Pa. In another embodiment, the anhydrous crystalline form has an unconfined yield strength from about 100 Pa to about 200 Pa at a major principal stress value of 500 Pa.

### Powder Time Consolidation Behavior Analysis

Solid forms prepared according to the procedures described for Routes E and F (Examples 13 and 14, respectively) were subjected to powder time consolidation behavior analysis in accordance with the following procedure.

A ring shear tester (RST-XS.s, Dietmar Schulze, Wolfenbüttel, Germany) with RST CONTROL 95 software was used for the flow function analysis. For this analysis, Low-Stress Shear Cell, XS-Lr0, bottom ring was over-filled with small portions of powder using a spatula. The excess powder was removed by gently scraping off the powder with the spatula until the powder was flush with the top of the ring. The bottom ring was weighed and the total mass was entered. When prompted the shear cell lid was attached to the loading rod and the filled bottom ring placed on the driving axle for the test. For the time consolidation test, a yield locus was first obtained by applying a 0.1 kPa preshear stress and 5 normal stresses equally spaced between 0 and the preshear stress to the powder and determining the corresponding shear stresses. A fresh powder sample was again prepared and conditioned to a similar steady state as that for the just obtained yield locus. The powder was held steady at 1000 Pa normal stress, and held constant for a specified duration of 12 hours. The powder was sheared again to failure and a new yield locus is drawn parallel to the previous yield locus but passing through the new shear point. From this yield locus a new unconfined yield strength is obtained by drawing a Mohr's circle tangential to the yield locus and passing through origin. The change in the shear stress obtained after this new conditioned state was determined and that represents the time consolidation behavior of the powder.

The results of this analysis are shown in FIG. 23.

It is well known that powders undergo consolidation when stored. At x=250 Pa on the graph of FIG. 23, the lower points represent the unconfined yield strength (UYS) at the initial time (0 hrs) and the higher points represent the values after storage (12 hrs). The strength gained by the powder as a result of storage is the time consolidation strength at a particular stress.

Practically, an active pharmaceutical ingredient will experience similar hold times in manufacturing settings (mixers, hoppers, storage bins, etc) and knowing the time consolidation behavior is important for anticipating flow issues.

The higher the UYS (y-axis) value after storage, the greater the impact and more likely flow issues will occur. Alternatively, the lower the unconfined yield strength the lower the consolidation tendency (better).

It can be seen from the results in FIG. 23 that the product of Route F (E14) does not exhibit as much of a consolidation effect as the product of Route E (E13).

1. A crystalline form of (5R)-5-(4-{[(2-fluorophenyl)methyl]oxy}phenyl)-L-prolinamide hydrochloride, characterised in that said crystalline form is an anhydrous form.

2. (canceled)

3. The crystalline form according to claim 1, which is selected from anhydrous form A (Form 1), anhydrous form B (Form 9) or anhydrous form C (Form 10).

4. The crystalline form according to claim 3, wherein the crystalline form is anhydrous form A (Form 1).

5. The crystalline form according to claim 3, wherein the anhydrous form A (Form 1) is characterized by an X-ray diffraction pattern having 2θ Diffraction (°) peaks at: 9.56±0.25, 12.71±0.25, 19.23±0.25, 20.40±0.25, 21.09±0.25, 21.47±0.25 and 27.37±0.25.

6. The crystalline form according to claim 3, wherein the anhydrous form A (Form 1) is characterized by the X-ray diffraction pattern of FIG. 2.

7. The crystalline form according to claim 3, wherein the crystalline form is anhydrous form B (Form 9).

8. The crystalline form according to claim 3, wherein the anhydrous form B (Form 9) is characterized by an X-ray diffraction pattern having 2θ Diffraction (°) peaks at: 16.33±0.25 and 21.86±0.25.

9. The crystalline form according to claim 3, wherein the anhydrous form B (Form 9) is characterized by the X-ray diffraction pattern of FIG. 18.

10. The crystalline form according to claim 3, wherein the crystalline form is anhydrous form C (Form 10).

11. The crystalline form according to claim 3, wherein the anhydrous form C (Form 10) is characterized by an X-ray diffraction pattern having 2θ Diffraction (°) peaks at: 17.48±0.25, 20.19±0.25, 21.76±0.25, 23.50±0.25 and 26.37±0.25.

12. The crystalline form according to claim 3, wherein the anhydrous form C (Form 10) is characterized by the X-ray diffraction pattern of FIG. 20.

13.-38. (canceled)

39. An anhydrous crystalline form of (5R)-5-(4-{[(2-fluorophenyl)methyl]oxy}phenyl)-L-prolinamide hydrochloride, characterized in that said anhydrous crystalline form has an initial bulk density, tested as defined herein, of at least 0.4 g/cm³.

40. An anhydrous crystalline form of (5R)-5-(4-{[(2-fluorophenyl)methyl]oxy}phenyl)-L-prolinamide hydrochloride, characterized in that said anhydrous crystalline form has an unconfined yield strength of less than 200 Pa at a major principal stress value of 500 Pa, tested in accordance with the powder flow function analysis herein.

41. A pharmaceutical composition comprising the crystalline form according to claim 1 with one or more pharmaceutically acceptable carrier(s), diluents(s) and/or excipient(s).

42.-44. (canceled)

45. A method of treating a disease or condition mediated by modulation of voltage-gated sodium channels which comprises administering a therapeutically effective amount of the crystalline form according to claim 1 to a subject in need thereof.

46. A crystalline form of (5R)-5-(4-{[(2-fluorophenyl)methyl]oxy}phenyl)-L-prolinamide hydrochloride, characterized in that said crystalline form is a solvated form.

47. The crystalline form according to claim 46, which is a form solvated with ethanol, methanol, 1-propanol, 1-butanol, 2-methoxyethanol, ethylene glycol or propylene glycol.
